SEQUENCE LISTING (21 sequences)

SEQ ID NO: 1 - DNA, 21 bp, synthetic construct
  source 1..21: mol_type = other DNA
gctggtcaaccggacacgcgg

SEQ ID NO: 2 - DNA, 24 bp, synthetic construct
  source 1..24: mol_type = other DNA
agggagccgataaagaggtcatcc

SEQ ID NO: 3 - DNA, 20 bp, synthetic construct
  source 1..20: mol_type = other DNA
ggctcatcgcagatcagaag

SEQ ID NO: 4 - DNA, 24 bp, synthetic construct
  source 1..24: mol_type = other DNA
ttatggacgaacgccgtgaaattg

SEQ ID NO: 5 - DNA, 22 bp, synthetic construct
  source 1..22: mol_type = other DNA
gccggattgtagttggtaggac

SEQ ID NO: 6 - DNA, 32 bp, synthetic construct
  source 1..32: mol_type = other DNA
ctgtctcagctgggaggcgacaaaaggccggc

SEQ ID NO: 7 - DNA, 39 bp, synthetic construct
  source 1..39: mol_type = other DNA
gaattagatccaccggtttgacgtctccgtgggcatcgg

SEQ ID NO: 8 - DNA, 60 bp, synthetic construct
  source 1..60: mol_type = other DNA
tgcccacggagacgtcaaaccggtggatctaattcaattagagactaattcaattagagc

SEQ ID NO: 9 - DNA, 43 bp, synthetic construct
  source 1..43: mol_type = other DNA
ccgatgctgtacttcttgtcggctgctgggactccgtggatac

SEQ ID NO: 10 - DNA, 24 bp, synthetic construct
  source 1..24: mol_type = other DNA
gacaagaagtacagcatcggcctg

SEQ ID NO: 11 - DNA, 21 bp, synthetic construct
  source 1..21: mol_type = other DNA
gtcgcctcccagctgagacag

SEQ ID NO: 12 - DNA, 31 bp, synthetic construct
  source 1..31: mol_type = other DNA
ctgcggcgatcgaaaggcaagggcattcagc

SEQ ID NO: 13 - DNA, 40 bp, synthetic construct
  source 1..40: mol_type = other DNA
cccgggcgagctcgcctaggctgtggacgccaaggagatg

SEQ ID NO: 14 - DNA, 59 bp, synthetic construct
  source 1..59: mol_type = other DNA
gctttcgatcgccgcagacgtcattttcaacgtcctcgatagtatagtggttagtatcc

SEQ ID NO: 15 - DNA, 18 bp, synthetic construct
  source 1..18: mol_type = other DNA
gcaccgactcggtgccac

SEQ ID NO: 16 - DNA, 46 bp, synthetic construct
  source 1..46: mol_type = other DNA
gtggcaccgagtcggtgcttttttgctcacctgtgattgctcctac

SEQ ID NO: 17 - DNA, 55 bp, synthetic construct
  source 1..55: mol_type = other DNA
cctaggcgagctcgcccggggatctaattcaattagagactaattcaattagagc

SEQ ID NO: 18 - DNA, 50 bp, synthetic construct
  source 1..50: mol_type = other DNA
gaggacgttgaaaatgacgtccggaactgcccatcacggccaaaagttcg

SEQ ID NO: 19 - DNA, 33 bp, synthetic construct
  source 1..33: mol_type = other DNA
ggcatccaagtatcgccatccgggatgcgactg

SEQ ID NO: 20 - DNA, 37 bp, synthetic construct
  source 1..37: mol_type = other DNA
acgtcattttcaacgtcctcgatagtatagtggttag

SEQ ID NO: 21 - DNA, 44 bp, synthetic construct
  source 1..44: mol_type = other DNA
gatggcgatacttggatgcccctatgcgagctcgcccggggatc

(19) United States
(12) Patent Application Publication
(10) Pub. No.: US 2024/0409959 A1
(43) Pub. Date: Dec. 12, 2024

(54) GENE DRIVE SYSTEM AND METHOD OF USE THEREOF

(71) Applicant: The Regents of the University of California, Oakland, CA (US)

(72) Inventors: Valentino Matteo GANTZ, La Jolla, CA (US); Victor Lauren LOPEZ DEL AMO, Pearland, TX (US); Xuechun FENG, Shenzhen (CN); Anthony JAMES, Irvine, CA (US)

(73) Assignee: The Regents of the University of California, Oakland, CA (US)

(21) Appl. No.: 18/697,145

(22) PCT Filed: Oct. 7, 2022
(86) PCT No.: PCT/US2022/077736

§ 371 Date: Mar. 29, 2024

(51) Int. Cl. C12N 15/90; A01K 67/033; C12N 9/22; C12N 15/11

(52) U.S. Cl. CPC C12N 15/902; A01K 67/0339; C12N 9/22; C12N 15/11; A01K 2217/07; A01K 2227/706; C12N 2310/20

(57) ABSTRACT

The present disclosure provides a novel gene drive methodology that can be used to spread engineered traits into a targeted population. More specifically, the present disclosure provides a method of using a transgenic line containing a modified Cas9, e.g., nickase Cas9, that introduces single-strand breaks/nicks, instead of double-stranded DNA cuts, to promote super-Mendelian inheritance of an engineered gene drive allele. The present disclosure further provides a Cx.quin. Cas9 (e.g., nickase Cas9) line and a Cx.quin. gene-drive line for suppression populations.

### CROSS-REFERENCE

This application claims the benefit of U.S. Provisional Application No. 63/254,753, filed Oct. 12, 2021, the entire content of which is incorporated herein by reference.

### STATEMENT REGARDING FEDERALLY SPONSORED RESEARCH OR DEVELOPMENT

This invention was made with government support under grant number OD023098 and AI162911 awarded by the National Institutes of Health. The government has certain rights in the invention.

### FIELD AND BACKGROUND

The present disclosure relates to gene drive system and method of use thereof. Traditional gene drives approaches employ a Cas9 that introduces DNA double-strand breaks to promote homology-directed repair (HDR) and consequent gene drive propagation. While the scientific community suggested the use of a nickase Cas9 for population engineering, no experimental data is available about the efficiency of a nickase Cas9 to edit an organism germline in a gene drive context. While paired nicks demonstrated their ability to disrupt genes and promote HDR in vitro there exists no system using a nickase Cas9 to introduce paired-nicks and promote HDR in the germline of a living organism.

The Culex pipiens species complex includes several species and their hybrid forms, including Cx. pipiens pipiens, Cx. pipiens pallens, Cx. pipiens molestus, Cx. quinquefasciatus, Cx. australicus, and Cx. globocoxitus. While some of these species have limited distribution, Cx. pipiens pipiens, Cx. quinquefasciatus, and their hybrids are widespread globally and represent the primary urban and suburban disease vector. The ability of these species to produce fertile hybrids has been a source of an ongoing discussion around the taxonomy of these species and has been proposed as a mechanism explaining the surprising adaptability of these mosquitoes and may account for the rapid evolution of insecticide resistance across the Culex pipiens complex. While CRISPR has allowed the development of engineered gene drives (GDs) based on homing endonucleases and other technologies that use genetically-engineered vectors to modify or suppress wild populations, Culex has been somewhat neglected regarding the development of genetic suppression strategies. No extensive CRISPR technology application has been achieved, mostly due to the fewer tools available for this species.

More background information related to gene drive system, and method of use thereof, fir genetic suppression, is described in EXAMPLES 1 and 2, respectively, of the present disclosure.

### SUMMARY

The present disclosure provides a novel gene drive methodology that can be used to spread engineered traits into a targeted population. The method of the present disclosure uses a transgenic line containing a modified Cas9 (i.e., nickase Cas9) that introduces single-strand breaks (nicks, instead of double-stranded DNA cuts) which promote super-Mendelian inheritance of an engineered gene drive allele. Further, the present disclosure provides a full sequence of a gene drive system, based on the use of nickase which is built for applications aimed at the suppression of Culex quinquefasciatus mosquitoes.

In certain embodiments, the present disclosure provides a system that is the first example of a gene drive that is able to bias Mendelian inheritance using a nickase-Cas9. In certain embodiments, a (modified) nickase Cas9 is utilized that introduces paired-nicks to produce a DNA break an allow the propagation of an engineered genetic element. The use of nicks instead of double-stranded breaks could lead to increased flexibility in gene drive design. Benefits include: 1) more stereotyped patterns of non-conversion alleles (insertion-deletions caused by the NHEJ DNA-repair pathway) which would allow to target larger beneficial portions of the gene target to ensure the negative selection of non-conversion alleles; and 2) the use of nickases should reduce the generation of mutation at off-target sites.

Several potential advantages using a nickase-based gene-drive system also include, first, DNA nicks are involved in important biological processes such as DNA replication and are typically repaired efficiently. For this reason, the novel arrangement of the method of the present disclosure could help limit the formation of resistant alleles; if paired-nicks do not occur simultaneously, single nicks should restore the original wildtype sequence to allow further targeting for gene drive conversion. Second, a nickase-based gene-drive approach could increase specificity and reduce off-target effects, which represent nonspecific genome editing events, as two independent cleavage events need to happen coordinately to obtain the desired modification. For example, gene drives also demonstrated its ability to bias Mendelian inheritance using mice, and a nickase-based gene-drive system should be translatable to these animals to improve specificity and reduce observed undesired error effects when editing mammalian embryos. Third, evidence is provided that the nH840A transgene combined with paired-gRNAs in a PAM-in configuration frequently generated large deletions between the spaced nicks; a scenario that is not detected when using the regular Cas9 inducing double-strand breaks. This property could be harnessed to boost efficiency when targeting vital genes or essential protein domains. Such large deletions should produce non-functional alleles resulting in non-viable animals in case of HDR failure, therefore ensuring the removal of individual escapees carrying small indels that allow for animal viability within a population.

Furthermore, only two out of four experimental conditions lead to successful gene drive, suggesting that the application of a nickase-Cas9 to gene drive was not obvious a priori. Additionally, only one of the two working conditions gives maximal efficiency, further supporting the uniqueness of the disclosed method.

The present disclosure further provides that the method and the nickase-based gene drive system of the present disclosure have several applications. For instance, adaptation of the system to generate nickase gene drive mosquitoes that could be used for population engineering to reduce the impact of mosquito-borne diseases. Using a nickase gene drive system in mice to trigger HDR while reducing off-target effects for field application to control pests or invasive species. A similar approach could also be applied in mammalian cells to boost HDR rates, while lowering off-targets.

The present disclosure also provides a full sequence of a gene drive system, based on the use of nickase which is built for applications aimed at the suppression of Culex quinquefasciatus mosquitoes. This is the first example of full-gene drive tailored for the suppression of this mosquito species. In certain embodiments, the present disclosure provides development strategies and approaches to generate the building blocks for the expression of CRISPR components, develop the know-how for site-specific transgenesis, and build a gene drive system for the suppression of Cx. quin. The present disclosure provides improvements that would obviate drawbacks of previous technology which aimed at the disruption of doublesex in females instead of the conversion to functional males. While the outlined strategy is tailored to this species (Culex quinquefasciatus), the same approach could be used for the suppression of other mosquitoes or insects (examples are Anopheles mosquitoes, Aedes mosquitoes, Drosophila suzukii fruit fly pest, etc.). The present disclosure provides both a graphical representation of the gene drive construct and the full sequence of the material.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Many aspects of the present disclosure can be better understood with reference to the drawings. The components in the drawings are not necessarily to scale, emphasis instead being placed upon clearly illustrating the principles of the present disclosure. Moreover, in the drawings, like reference numerals designate corresponding parts throughout the several views. A better understanding of the features and advantages of the invention will be obtained by reference to the following detailed description that sets forth illustrative embodiments, in which the principles of the invention are utilized, and the accompanying drawings (also “Figure” and “FIG.” herein), of which:

FIGS. 1A-1E. A nickase-based gene drive system promotes different overhang patterns. FIG. 1A. Schematic diagram of a traditional CopyCat gene-drive system. When combined with a Cas9 source, the gRNA cassette replaces the wildtype allele (converted allele) by DNA double-strand break and subsequent homology directed-repair (HDR). FIG. 1B. A nickase Cas9 source is combined with a Copycat containing two gRNAs targeting each complementary strand of the wildtype allele to spread the paired gRNA cassette by HDR. FIG. 1C. Wildtype Cas9 cuts both DNA strands, nD10A cuts the target strand where the gRNA is bound, and nH840 cuts the non-target strand. FIG. 1D. Design of the paired gRNAs in both PAM-out and PAM-in orientation. Paired gRNAs target sites are located ˜50 nucleotides apart. The depicted gRNAs bind to the opposite strand when produced by complementarity. PAM sequences (not included in the gRNA) are crucial for DNA recognition. The triangles denote the different cut sites associated with each gRNA FIG. 1E. Wildtype Cas9 introduces blunt ends when combined with either of the CopyCat elements. nD10A and nH840, combined with paired gRNAs binding to specific DNA strands, can generate 5′ or 3′ overhangs as they target different strands (target and non-target strands, respectively).

FIGS. 2A-2E. Super-Mendelian inheritance rates produced by nickase Cas9s when 5′ overhangs are generated. FIG. 2A. All Cas9 sources (wildtype Cas9, nD10A, and nH840A) and the CopyCat elements are inserted in the X chromosome (yellow [y] and white [w] genes, respectively). F0 males containing the Cas9 were crossed to females containing either Copycat gene drives (CC-GD). F1 females carrying both transgenes were crossed to wildtype males to assess germline allelic conversion (triangle indicates potential wildtype allele replacement) by scoring the GFP marker in the F2. FIG. 2B. Similar biased inheritance rates were observed when wildtype Cas9 was combined with both CopyCat elements. FIG. 2C. nD10A and nH840A triggered super-Mendelian inheritance rates only when generating 5′ overhangs. FIG. 2D. Schematic of observed resistant allele outcomes in the gene-drive experiments. FIG. 2E. Cas9 PAM-out (n=20) displayed large insertions while Cas9 PAM-in (n=18) produced high rates of simultaneous mutations occurring at both target sites. nD10A (n=20) produced a high frequency of large insertions while nH840A (n=24) produced bigger deletions between nick sites.

FIGS. 3A-3C. Allelic conversion (HDR), mutations (resistant alleles) and wildtype (WT; uncut or in-frame mutations) rates produced by the regular Cas9 and nickase versions combined with the CopyCat elements. This analysis represents the outcomes produced by the wildtype Cas9 and nickase versions when combined with the white CopyCat experiments in FIGS. 2b & 2c. FIG. 3A. Experimental cross with alleles present in the experimental design are depicted. Yellow and white genes are located 1.5 centimorgans in the X chromosome of Drosophila. The chromosome containing the Cas9 sources (inserted in yellow), and which (wildtype) white gene is targeted by the gene-drive element for conversion in F1 females, represents the receiver chromosome. The CopyCat element (CC-GD) is inserted in white, representing the donor chromosome (chromosome of origin of the gene-drive element). FIG. 3B. The results were graphed according to three possible categories based on the phenotypic readouts: 1) Allelic conversion or HDR—alleles that were converted (presenting as DsRed+, GFP+ and white eye), 2) resistant allele events—alleles that were cut but were not converted (presenting as DsRed+, GFP− and white eye), and 3) wildtype—these individuals displayed wildtype eyes, DsRed+ and GFP−, suggesting that these alleles were not acted upon. FIG. 3C. The cutting efficiency ([conversion+NHEJ]/total) for wildtype Cas9 was ˜100% when using both gene-drive CopyCat elements. Wildtype/uncut alleles were almost not detected, Cas9 combined with PAM-out showed only 1% uncut alleles while Cas9 PAM-in displayed 0% uncut alleles. Considering the conversion efficiency, both Cas9 situations showed 94-95% HDR rates. nD10A generating 5′ overhangs produced 85% conversion, 10% mutations and 5% wildtype of the targeted alleles. nH840A generating 5′ overhangs produced 70% conversion rates and 21% mutations, wildtype alleles represented 9% in this situation. nD10A and nH840A producing 3′ overhangs did not trigger HDR. In this same condition where nickases generated 3′ overhangs, nD10A only induced 3% cutting and 97% wildtype alleles. nH840A produced ˜41% cutting and 59% wildtype/uncut alleles.

FIGS. 4a-4e. Gene drive mechanism and inheritance. A GD uses encoded Cas9 and gRNA to cut the wild-type allele (FIG. 4a), then, by means of the HDR pathway the construct gets copied onto the broken allele (FIG. 4b), which results in homozygous condition for the drive element (FIG. 4c). This action bypasses traditional inheritance (FIG. 4d), as when this process happens in the germline, it leads to super-Mendelian inheritance (FIG. 4e), which can be used to spread engineered traits to modify or suppress entire wild populations.

FIGS. 5a-5d. Constructs and testing. FIG. 5a. Four plasmids each carrying a construct for expressing Cas9. FIG. 5b. Constructs for expression of gRNAs. FIG. 5c. First step for testing the constructs by co-injecting each Cas9 construct with a mix of all the four gRNAs. FIG. 5d. The highest-expressing ubiquitous-Cas9 was used to validate the gRNA constructs in FIG. 5b by co-injecting it with each of them.

FIGS. 6a-6d. Constructs. FIG. 6a. Ubiquitous Cas9 transgene marked with DsRed, to be inserted in the white locus. FIG. 6b. Opie2>DsRed. FIG. 6c. GD construct to be inserted in the dsx locus. FIG. 6d. IE1>GFP transient expression in 2nd instar larvae. White arrows point at single larvae in FIG. 6b & 6d.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present disclosure provides a novel gene drive method and Cas9 nickase-based gene drive system that can be used to spread engineered traits into a targeted population. In certain embodiments, the method of the present disclosure uses a transgenic line containing a modified Cas9 (e.g., nickase Cas9) that introduces single-strand breaks (nicks, instead of double-stranded DNA cuts) which promote super-Mendelian inheritance of an engineered gene drive allele. A full sequence of a gene drive system, based on the use of nickase which is built for applications aimed at the suppression of Culex quinquefasciatus mosquitoes is also provided. The present disclosure further provides building blocks for the expression of CRISPR components, develop the know-how for site-specific transgenesis, and build a gene drive system for the suppression of Cx. quin.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### A Nickase Cas9 Gene-Drive System Promotes Super-Mendelian Inheritance in Drosophila

### Introduction

CRISPR gene-drive systems have emerged as a promising tool for disseminating engineered traits into wild populations to control disease transmission. This rapid dissemination is possible due to the ability of these systems to surpass Mendel's First Law of gene segregation, which dictates that an allele has a 50% probability of being passed to the next generation; in fact, gene drives can reach up to 100% inheritance of a desired allele. A proof-of-concept system was first implemented in flies (Gantz and Bier, 2015) and was applied to different mosquitoes such as Anopheles or Aedes under laboratory conditions to fight vector-borne diseases (Adolfi et al., 2020; Gantz et al., 2015; Hammond et al., 2016; Kyrou et al., 2018; Li et al., 2020; Simoni et al., 2020).

CRISPR-based gene drives consist of a three-component transgene: (i) Cas9, a DNA nuclease that produces DNA double-strand breaks; (ii) a guide RNA (gRNA) that directs Cas9 to cleave the DNA at a predetermined site; and (iii) two homology arms flanking the Cas9/gRNA components that perfectly match both sides of the cut site to promote homology-directed repair (HDR). When a gene-drive individual mates with a wildtype, the encoded Cas9 from the engineered gene drive cuts the wildtype allele in the germline, which is replaced by HDR using the intact gene-drive chromosome as a repair template. With the gene drive present on both alleles (i.e., homozygous), this process produces a super-Mendelian inheritance (>50%) of the engineered cassette to spread new traits through a population.

Current gene-drive methods employ a Cas9 that introduces DNA double-strand breaks (Adolfi et al., 2020; Bier, 2021; Gantz et al., 2015; Hammond et al., 2016; Kyrou et al., 2018; Li et al., 2020; Simoni et al., 2020). However, lack of alternative Cas9-based strategies could enlarge the available toolkit for gene-drive designs while potentially bringing advantages to improve the current ones. In fact, mutant versions of Cas9 that only generate nicks should also be amenable. Wildtype Cas9 contains two endonuclease domains (HNH and RuvC-like domains) that can introduce DNA double-strand breaks, where each cleaves one strand of the DNA double-helix (Gasiunas et al., 2012; Jinek et al., 2012). By mutating critical residues in the nuclease domains, two nickase versions of Cas9 can be generated: i) nCas9-D10A (nD10A) contains an inactivated RuvC domain and only cuts the target strand where the gRNA is bound, and ii) nCas9-H840A (nH840A) contains an inactivated HNH and so only cuts the non-target strand (Gasiunas et al., 2012; Jinek et al., 2012).

Importantly, the nickase versions of Cas9 already demonstrated their efficiency for genome editing. nD10A has been used to generate paired DNA nicks and efficiently disrupts genes in Drosophila and cell culture (Gopalappa et al., 2018; Port et al., 2014). Nicks induced by nD10A promoted higher HDR rates than nH840A, where HDR was almost undetectable (Bothmer et al., 2017; Hyodo et al., 2020; Mali et al., 2013; Wang et al., 2021, 2018). Furthermore, nD10A can boost specificity while reducing off-target effects since it requires two gRNAs that target complementary DNA strands to either disrupt a gene function or trigger HDR. When the same pair of gRNAs were combined with wildtype Cas9, undesired off-target effects at an unspecific genomic region were generated, which does not occur with nD10A (Ran et al., 2013).

Nickase Cas9 (nCas9) versions have been extensively utilized in vitro to evaluate HDR efficiencies. However, only a few studies attempted to employ paired-gRNAs' nickase-based approaches using mice and Drosophila, where nD10A promoted modest HDR rates (Lee and Lloyd, 2014; Ren et al., 2014). Therefore, the design of novel strategies using both nD10A and nH840A to induce meaningful HDR rates in the germline of a living organism is needed to better understand the fine-workings of nickase-based HDR in vivo. A nCas9-based gene drive might be applicable for population engineering to bring potential advantages. For example, DNA nicks are involved in important biological processes such as DNA replication and are typically repaired efficiently (Caldecott, 2008; Chafin et al., 2000; Reyes et al., 2021; Wang and Hays, 2007). Therefore, if paired nicks do not occur simultaneously, single nicks could restore the original wildtype sequence, reducing the formation of mutations or resistant alleles at the target site for further gene-drive conversion. Additionally, DNA nicks follow distinct DNA repair pathways compared to DNA double-strand breaks that are introduced by traditional gene drives (Vriend and Krawczyk, 2017), and the intrinsically offset distance between paired nicks in a gene-drive setting could potentially favor the formation of specific mutations to improve gene-drive propagation in certain applications.

Thus, simultaneous paired nicks targeting two adjacent DNA regions should generate a staggered double-strand break followed by DNA repair by HDR to promote super-Mendelian inheritance of an engineered gene-drive construct. Herein, a nCas9-based gene-drive system was developed which promoted super-Mendelian inheritance in Drosophila melanogaster as a proof-of-concept and showed that nD10A and nH840A can promote efficient HDR in the germline. Interestingly, it showed that super-Mendelian inheritance rates can be achieved only when the gene-drive design generated 5′ overhangs. It also showed that nH840A produces larger deletions compared to nD10A when the allelic conversion fails. This feature can potentially be employed to reduce the presence of viable animals carrying small mutations when targeting essential genes, as strategies to generate large deletions removing critical protein domains to ensure insects' lethality could be designed. Overall, this work expands the technology and applicability of CRISPR gene-drive systems for genetic engineering of wild populations.

### Experimental Model and Subject Details

### Fly Rearing and Crosses

All flies were kept on standard food with a 12/12 hours day/night cycle. Fly stocks are kept at 18° C., and all experimental crosses were performed at 25° C. All flies were anesthetized during the experiments using CO₂. F₀ crosses from gene-drive experiments were made in pools of 3-6 virgin females crossed to 3-6 males. F1 experiments were always made in single pairs to track editing events happening singularly in the germline. The F2 progeny was scored as male or female and sorted for a fluorescent marker (DsRed and/or GFP) using a Leica M165 F2 Stereomicroscope with fluorescence as an indicator of transgene inheritance rates. All experiments were performed in a high-security ACL2 (Arthropod Containment Level 2) facility built for gene drive purposes in the Division of Biological Sciences at the University of California, San Diego. Crosses were made in polypropylene vials (Genesee Scientific Cat. #32-120), and all flies were frozen for 48 hours before being removed from the facility, autoclaved, and discarded as biohazardous waste.

### Method Details

### Plasmid Construction

DNA constructs were built using NEBuilder HiFi DNA Assembly Master Mix (New England BioLabs Cat. #E2621) and transformed into NEB 10-beta electrocompetent E. coli (New England BioLabs Cat. #3020). DNA was extracted using a Qiagen Plasmid Midi Kit (Qiagen Cat. #12143) and sequenced by Sanger sequencing at Genewiz. Primers used for cloning can be found in the Key Resources Table below.

### Transgenic Line Generation and Genotyping

Embryo injections were outsourced to Rainbow Transgenic Flies, Inc. All DNA constructs were injected into the lab's isogenized Oregon-R (Or-R) strain to ensure consistent genetic background throughout experiments. Plasmid templates were co-injected with a Cas9-expressing plasmid (pBSHsp70-Cas9 was a gift from Melissa Harrison & Kate O'Connor-Giles & Jill Wildonger [Addgene plasmid #46294; RRID: Addgene_46294]). The injected generation 0 (G₀) animals were received and then intercrossed the hatched adults in small pools (3-5 males×3-5 females), and the G₁ flies were screened for a fluorescent marker (DsRed for Cas9 versions and GFP for gene-drive elements, both fluorescences in the eye), which was indicative of transgene insertion. Homozygous lines were established from single transformants by crossing to Or-R. As the Cas9 transgene is inserted into the yellow gene disrupting it, homozygous flies for the Cas9 versions can be identified once flies display a yellow body color. Similarly for the Copycat constructs that are integrated into the white gene, homozygous flies for the CopyCat elements display a white eye phenotype. Stocks were sequenced by PCR and Sanger sequencing to confirm proper transgene insertion.

DNA Extraction from Single Flies

To sequence resistant alleles, genomic DNA was extracted from individual males following the method described by Gloor GB and colleagues (Gloor et al., 1993). In brief, 50 ul of the extraction buffer was used to squish single flies in a PCR tube which was placed into the PCR machine (Proflex PCR system, Applied Biosystems) for 1 hour at 37° C. followed by 5 minutes at 95° C. to inactivate the proteinase K. Then, 200 μL of water was added to each DNA sample to obtain a total of 250 uL per sample. 1-5 uL was used in a 25 uL PCR reaction covering the gRNA cut sites in the white gene for Sanger sequencing analysis.

### Sanger Sequencing of Individuals Carrying Resistance Alleles

A DNA region covering the gRNA cut sites was amplified using the v1564 and v1565 oligos (see oligos list below). The obtained amplicon was then sequenced by Sanger sequencing to determine the quality of the resistant alleles using the v478 oligo. When lower-quality traces were obtained, a second Sanger sequencing reaction was performed from the other side of the amplicon to confirm the quality of the mutation with either the v659 or v1571 primers. Primers used for resistance allele sequencing are listed in the following Table.

| | | | |
|---|---|---|---|
| | | | SEQ ID |
| REAGENT or RESOURCE | SOURCE | IDENTIFIER | NO: |

Oligonucleotides

| | | | |
|---|---|---|---|
| Primer for resistant allele sequencing: | This work | v478 | 1 |
| GCTGGTCAACCGGACACGCGG | | | |
| Primer for resistant allele sequencing: | This work | v659 | 2 |
| AGGGAGCCGATAAAGAGGTCATCC | | | |
| Primer for resistant allele sequencing: | This work | v1571 | 3 |
| GGCTCATCGCAGATCAGAAG | | | |
| Primer for resistant allele sequencing: | This work | v1564 | 4 |
| TTATGGACGAACGCCGTGAAATTG | | | |
| Primer for resistant allele sequencing: | This work | v1565 | 5 |
| GCCGGATTGTAGTTGGTAGGAC | | | |
| Primer for nickase constructs: | This work | v1146 | 6 |
| CTGTCTCAGCTGGGAGGCGACAAAAGGCCGGC | | | |
| Primer for nickase constructs: | This work | v1144 | 7 |
| GAATTAGATCCACCGGTTTGACGTCTCCGTGGG | | | |
| CATCGG | | | |
| Primer for nickase constructs: | This work | v1145 | 8 |
| TGCCCACGGAGACGTCAAACCGGTGGATCTAAT | | | |
| TCAATTAGAGACTAATTCAATTAGAGC | | | |
| Primer for nickase constructs: | This work | v0725 | 9 |
| CCGATGCTGTACTTCTTGTCGGCTGCTGGGACT | | | |
| CCGTGGATAC | | | |
| Primer for gene-drive CopyCat constructs: | This work | v0724 | 10 |
| GACAAGAAGTACAGCATCGGCCTG | | | |
| Primer for gene-drive CopyCat constructs: | This work | 0377 | 11 |
| GTCGCCTCCCAGCTGAGACAG | | | |
| Primer for gene-drive CopyCat constructs: | This work | 611 | 12 |
| CTGCGGCGATCGAAAGGCAAGGGCATTCAGC | | | |
| Primer for gene-drive CopyCat constructs: | This work | v0992 | 13 |
| CCCGGGCGAGCTCGCCTAGGCTGTGGACGCCAA | | | |
| GGAGATG | | | |
| Primer for gene-drive CopyCat constructs: | This work | 615 | 14 |
| GCTTTCGATCGCCGCAGACGTCATTTTCAACGT | | | |
| CCTCGATAGTATAGTGGTTAGTATCC | | | |
| GCACCGACTCGGTGCCAC | This work | v0689 | 15 |
| Primer for gene-drive CopyCat constructs: | This work | v1015 | 16 |
| GTGGCACCGAGTCGGTGCTTTTTTGCTCACCTG | | | |
| TGATTGCTCCTAC | | | |
| Primer for gene-drive CopyCat constructs: | This work | v0973 | 17 |
| CCTAGGCGAGCTCGCCCGGGGATCTAATTCAAT | | | |
| TAGAGACTAATTCAATTAGAGC | | | |
| Primer for gene-drive CopyCat constructs: | This work | v1016 | 18 |
| GAGGACGTTGAAAATGACGTCCGGAACTGCCCA | | | |
| TCACGGCCAAAAGTTCG | | | |
| Primer for gene-drive CopyCat constructs: | This work | 612 | 19 |
| GGCATCCAAGTATCGCCATCCGGGATGCGACTG | | | |
| Primer for gene-drive CopyCat constructs: | This work | 1067 | 20 |
| ACGTCATTTTCAACGTCCTCGATAGTATAGTGG | | | |
| TTAG | | | |
| Primer for gene-drive CopyCat constructs: | This work | v1017 | 21 |
| GATGGCGATACTTGGATGCCCCTAIGCGAGCTC | | | |
| GCCCGGGGATC | | | |

### Microscopy

Adult flies were anesthetized using 002 to select individuals for crossing experiments. Their phenotypes were analyzed using a Leica M165 FC Stereo microscope to properly prepare the experimental crosses. Inheritance analysis of the transgenes marked with fluorescence was evaluated using the same microscope. DsRed marker implies presence of the Cas9 cassettes while GFP fluorescence indicates presence of the CopyCat transgenes.

### Quantification and Statistical Analysis

### Statistical Analysis

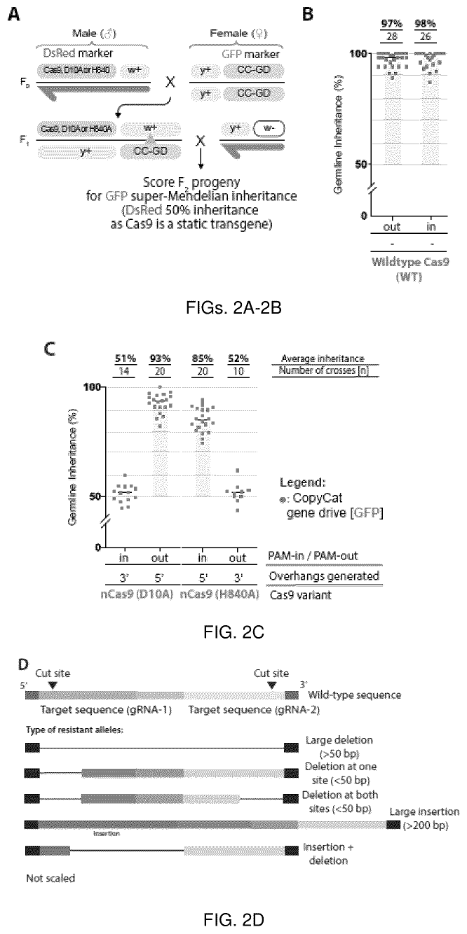

GraphPad Prism 9 and Adobe Illustrator were used to generate all the graphs. Statistical analyses were performed using GraphPad Prism 9. In FIG. 2B, an unpaired t test was applied to compare inheritance rates when using the wildtype Cas9. Additionally, One-Way Anova and Tukey's multiple comparison test to evaluate differences between super-Mendelian rates in the nickase gene-drive experiments in FIG. 2C. For evaluating the differences in proportions for resistant allele events in FIG. 2E, Fisher's exact test was used.

### Results

### A. Nickase Gene-Drive System can be Tailored to Induce 5′ or 3′ Overhangs

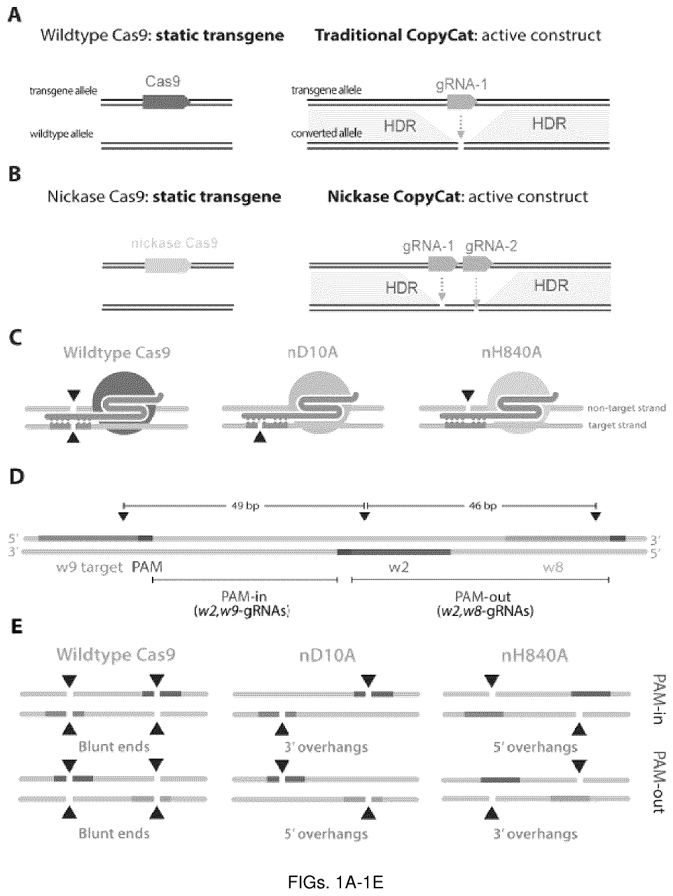

To design a nickase-based gene drive, a gRNA-only split-drive system (i.e., Copycat) was used that consists of two separate components: (i) a transgenic fly carrying a static Cas9 transgene, which is inherited in a Mendelian fashion, and (ii) an engineered animal carrying a CopyCat cassette formed by a gRNA gene that is flanked by two homology arms (Gantz and Bier, 2016). Once the two components are genetically crossed, traditional CopyCat gene drives rely on DNA double-strand breaks produced by a single gRNA, which targets the same sequence on the wildtype allele where it is inserted, to propagate the synthetic cassette by HDR (Champer et al., 2019; Gantz and Bier, 2016; López Del Amo et al., 2020a; Xu et al., 2017) (FIG. 1A). In contrast, the nickase-based gene-drive system requires two gRNAs since this modified Cas9 introduces nicks instead of double-strand breaks. In this case, the gRNA pair will produce two independent cleavage events on each of the complementary DNA strands for gene drive propagation by HDR, emulating traditional gene drives (FIG. 1B).

To build a gene drive system based on a nCas9, two transgenic lines containing a DsRed marker and carrying either the nD10A or nH840A versions were generated, which cut the target strand (bound to the gRNA) or the non-target strand, respectively. Additionally, a validated wildtype Cas9 line was employed, which introduces DNA double-strand breaks (López Del Amo et al., 2020b) as a positive control (FIG. 1C). All Cas9 transgenes were inserted into the yellow locus and expressed with the same vasa germline promoter. Separately, two Copycat gene-drive constructs were built and inserted into the white gene to produce two distinct gRNAs. To ensure perfect homology and a proper HDR process, the two homology arms included as part of the CopyCat elements match each cut site of the gRNA pair. A GFP marker was used to track the inheritance of these transgenes. Both Copycat lines share the w2-gRNA, which were previously validated (Lopez Del Amo et al., 2020a, 2020b), and were combined with either the w8-gRNA or the w9-gRNA as the second gRNA (FIG. 1D). Additionally, PAM DNA sequences are crucial for target location recognition (Jinek et al., 2012), and these constructs present different PAM orientations depending on the gene-drive element. The Copycat transgenic line containing w2,w8-gRNAs pairs (CC(w2,w8)) have PAMs that are facing in opposite directions (i.e., a PAM-out orientation). In contrast, the transgenic strain carrying the w2,w9-gRNAs pairs (CC(w2,w9)) have PAMs that are facing each other (i.e., a PAM-in orientation). All three gRNAs are located within a ˜100 base pair DNA window, and the paired gRNA cut sites are separated by approximately 50 nucleotides (FIG. 1D).

The nickases were combined with the two CopyCat transgenic lines to create four schemes to test the nickase gene-drive system: i) nD10A with the CC(w2,w9) (PAM-in) to generate 3′ overhangs; ii) nD10A with the CC(w2,w8) (PAM-out) to generate 5′ overhangs; iii) nH840A with CC(w2,w9) (PAM-in) to generate 5′ overhangs and iv) nH840A with the CC(w2,w8) (PAM-out) transgenic line to generate 3′ overhangs. Importantly, combining either of the CopyCat lines with wildtype Cas9 produces similar blunt ends in both situations (FIG. 1E).

### B. Cas9 Nickases Promote Super-Mendelian Inheritance of the CopyCat Elements

To evaluate the efficiency of the CopyCat elements, the same genetic cross scheme was used in all cases. First, male flies containing the Cas9 source (wildtype Cas9, nD10A, or nH840A) were combined with the CopyCat lines to obtain F1 trans-heterozygous animals carrying both transgenes. Then, these F1 females were crossed to a white mutant line to evaluate biased inheritance in their F2 progeny. If the Copycat is inactive, 50% inheritance of the GFP marker should be observed, which is integrated within the engineered cassette as mentioned. If the CopyCat construct can promote HDR in the germline, it should display super-Mendelian inheritance (>50%) of the GFP-marked transgene. All Cas9 sources, which carry the DsRed marker, should have ˜50% inheritance (FIG. 2A).

When combining the wildtype Cas9 with both CopyCat elements, two proximal DNA double-strand breaks were introduced by a multiplexing approach (FIG. 1E), which efficiently biases Mendelian inheritance (Champer et al., 2018). Here, similar super-Mendelian inheritance levels of ˜97% were observed with both PAM-out and PAM-in CopyCat elements (FIG. 2B). No significant differences between CopyCat elements was observed when combined with the wildtype Cas9 (p=0.6721), suggesting that both gRNA combination pairs would have similar efficiencies as a unit. In previous work, the w2-gRNA in a similar CopyCat arrangement that showed 90% super-Mendelian inheritance was individually validated (López Del Amo et al., 2020a). Here, adding a second gRNA (w8 or w9) boosted the allelic conversion efficiency from 90% to ˜97% in both cases. While these results concur with reports of increased gene-drive performance with an additional gRNA (Champer et al., 2018), the contribution of each gRNA singularly in a multiplexing approach cannot be determined due to different cleavage window activities and PAM orientations between gRNA pairs.

After confirming the activity of the two elements built to test the nickase gene drive, the ability of nCas9 to promote super-Mendelian inheritance in either of the four defined scenarios was evaluated (FIG. 1E). First, the nD10A transgenic line was crossed with both CopyCat strains carrying the tandem gRNAs following the same experimental cross scheme (FIG. 2A). In this case, the nD10A version displayed 93% super-Mendelian inheritance levels when combined with the CC(w2, w8) that generated 5′ overhangs. In contrast, the CC(w2, w9) (PAM-in) that generated 3′ overhangs was inherited in a Mendelian fashion (˜50%) (FIG. 2C).

The nH840A line following the same cross scheme was also tested (FIG. 2A). nH840A produced super-Mendelian inheritance rates of ˜85% when combined with the CC(w2, w9) (PAM-in) that generated 5′ overhangs. However, 50% inheritance rates were observed when combined with the CC(w2, w8) (PAM-out) to generate 3′ overhangs (FIG. 2C). Interestingly, super-Mendelian inheritance was observed when using both nD10A and nH840A, yet nD10A produced biased inheritance only when combined with the CC(w2, w8) while nH840A triggered super-Mendelian inheritance when crossed to the CC(w2, w9). Therefore, gene-drive activity was detected only when 5′ overhangs were generated, and these results concurred with previous in vitro studies where paired nicks only stimulated significant HDR levels when using nD10A to generate 5′ overhangs (Mali et al., 2013; Ran et al., 2013).

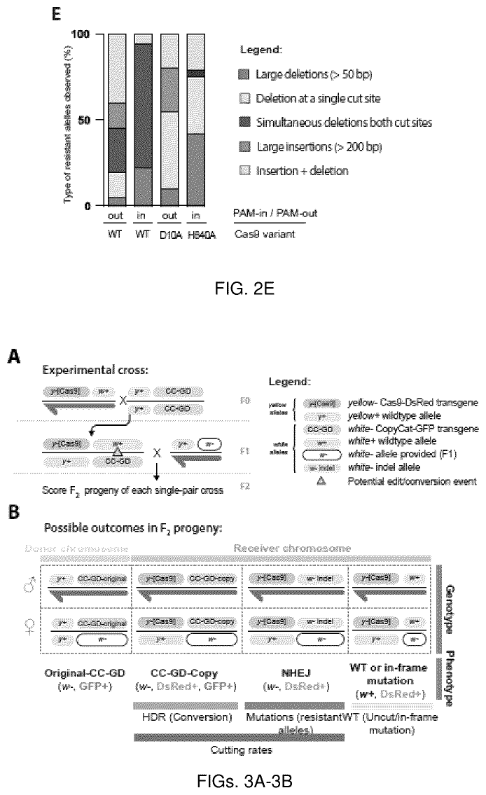

Overall, nD10A performed significantly better than nH840A for inheritance bias when generating 5′ overhangs (p<0.0001), which could be due to different cleavage rates between the nickases. Since the white gene targeted for conversion in this system is tightly linked with the yellow gene where all the Cas9 sources are inserted, this experimental design allowed us to evaluate conversion (HDR), mutations (resistant alleles) and wildtype/uncut allele rates arising from all conditions tested via the F2 progeny (FIGS. 3A-3C). nD10A displayed 95% cutting efficiency, which is higher than nH840A that showed 91% cutting rates (p=0.0683; FIGS. 3A-3C). It was also observed that nD10A displayed higher conversion rates (85%) compared to the nH840A (70%). Most importantly, nickases producing biased inheritance contained ˜5-10% wildtype alleles, suggesting that DNA nicks can restore the original wildtype sequence to allow further gene-drive conversion (FIGS. 3A-3C).

Altogether, it was demonstrated the example of a gene-drive system driven by a nCas9, which simultaneously nicks both complementary strands to induce efficient allelic conversion that is mediated by HDR in vivo. Furthermore, these data indicate that super-Mendelian inheritance through nickase gene drives can only be achieved by generating 5′ overhangs.

DNA double-strand breaks introduced by gene drives can produce resistant alleles at the target site when the HDR-mediated allelic conversion process is inaccurate. Resistant alleles generated by wildtype Cas9 have been characterized (Champer et al., 2018; Gantz and Bier, 2015; Hammond et al., 2017; Lopez Del Amo et al., 2020b). However, the types of resistant alleles generated by paired nicks are uncharacterized in a gene-drive context. Here, the resistant alleles generated by the wildtype Cas9 and nickase gene-drive systems were identified and evaluated, providing promoted super-Mendelian inheritance (FIGS. 2B-2C). Since the CopyCat target is the white gene located in the X chromosome, and males have only one X chromosome, GFP-negative F2 males that present the white eye phenotype indicate white gene disruption and unsuccessful allelic conversion, indicating individuals that carry resistant alleles.

The DNA was extracted from these animals and characterized by Sanger sequencing. Five different classes of resistant alleles were observed: i) large deletions spanning both cut sites (>50 base pairs [bp]), ii) deletions (<50 bp) occurring at one cut site with the other cut site intact, iii) simultaneous deletions (<50 bp) at both target sites with partial sequence retention between cut sites, iv) large insertions (>200 bp) and v) small insertions combined with small deletions in the same individuals at the same cut site (i.e., insertion+deletion) (FIG. 2D).

When F2 flies arising from the nH840A and PAM-in gRNAs combination that carried resistant alleles were sequenced, it was detected that 42% of these individuals contained large deletions spanning 50 to 90 nucleotides (FIG. 2E). This is a significantly higher frequency of large deletions spanning both nick sites compared to the nD10A PAM-out (5%) (FIG. 2E; p=0.0389). Similarly, wildtype Cas9 combined with PAM-out and PAM-in gRNAs displayed a lower percentage of large deletions, ˜5% and ˜20% respectively, compared to the nH840A PAM-in combination (FIG. 2E).

Large insertions (>200 bp) were also observed when nD10A was combined with paired gRNAs in a PAM-out orientation. It was found that 25% of the sequenced flies within this condition had large insertions, which was significantly higher than nH840A combined with PAM-in gRNAs, where no large insertion was detect (FIG. 2E; p=0.0143). In line with these observations, the wildtype Cas9 only produced large insertions when combined with the PAM-out gRNAs, suggesting that PAM orientation can influence resistant allele outcomes (FIG. 2E). The large insertions produced by the PAM-out gRNAs represented partial HDR occurrences containing portions of the engineered gene-drive allele. Either part of the U6 promoter downstream of the left homology arm or the synthetic 3×P3 promoter with an incomplete piece of the GFP marker downstream of the right homology arm of the gene-drive element was detected. While previous gene drive studies showed the formation of insertions with either a multiplexing approach (Champer et al., 2018) or traditional gene drives with a single gRNA (Hammond et al., 2017), this outcome provides a more prominent phenomenon of the PAM out arrangement in this work.

No significant differences between the nickase genotypes was observed in the other categories described above, including single cuts at only one target site, simultaneous mutations at both cut sites or insertion+deletion events happening in the same individual (FIGS. 2D-2E). However, it is noteworthy that a significant percentage of simultaneous small mutations occurred at both cut sites when using wildtype Cas9 with PAM-out and PAM-in gRNAs, 25% and 72%, respectively. Interestingly, these occurrences were almost undetectable when the gene-drive elements were combined with nickases (FIG. 2E). Instead, both nickases presented higher rates of resistant alleles containing mutations at only one target site at a time (FIG. 2E). This may indicate that DNA double-strand breaks induce small deletions if allelic conversion fails while DNA nicks can restore the wildtype sequence. Indeed, this aligns with the presence of wildtype alleles (uncut) when using nickase versions of Cas9 (FIGS. 3A-3C).

Overall, it is confirmed that the gRNAs in this work are active, as mutations were found at all target sites within the genotypes that produced super-Mendelian inheritance. Additionally, different repair outcomes were also shown when using distinct Cas9 sources, which can inform future gene-drive strategies.

### Discussion

In this work, it reports a gene-drive system based on nickase versions of Cas9 that promotes super-Mendelian inheritance in Drosophila and increases the number of feasible design options for gene drives aimed at population engineering. It showed that both nD10A and nH840A produced efficient HDR in the germline, but only when the two nicks in the DNA generated 5′ overhangs. Characterization of events that failed to convert the wildtype allele to the gene drive by Sanger sequencing indicated that nH840A combined with PAM-in gRNAs produced higher rates of large deletions compared to the nD10A and PAM-out arrangement. While the PAM-out condition triggered large insertions, it was not observed such alterations when resistant alleles produced by the nH840A were analyzed, suggesting that the modes of DNA repair that are triggered are nickase dependent.

In this Example reported herein, nD10A produced higher super-Mendelian rates than nH840A, which may attribute to differences in cleavage activities between the nickases. In fact, nH840A has been shown to present less cleavage activity in vitro as it produced lower indel rates when disrupting the EMX-S1 gene (Gopalappa et al., 2018). Additionally, the distinct time-windows of cleavage between the pairs of gRNAs, which need to cut simultaneously, may impact HDR efficiencies as both nickases showed super-Mendelian inheritance with different paired gRNAs. While previous studies did not report meaningful HDR rates with nH840A (Bothmer et al., 2017; Mali et al., 2013; Ran et al., 2013), here, it showed efficient HDR rates achieved by gRNAs in a PAM-in configuration when generating 5′ overhangs using nH840A for the first time. Thus, nH840A could be a viable option for future nickase-based designs to promote HDR.

While the focus here was for a Cas9-nickase gene drive, slightly higher super-Mendelian rates was observed in the wildtype Cas9 over the nickases. This is because a nickase requires the coordinated action of both gRNAs cutting simultaneously to induce efficient HDR. Conversely, when the wildtype Cas9 is used, a single cut from either of the paired gRNAs can induce HDR to produce double-stranded DNA breaks. Furthermore, while failure of the first gRNA would result in a small indel, the second gRNA can still cut and trigger a second round of potential conversion, and which could explain the higher inheritance rates in the wildtype Cas9 scenario. Thus, thoroughly testing the paired gRNAs to maximize coordinated action in nickase gene-drive systems could be important.

With regard to resistant allele formation, it was shown that the nH840A transgene combined with paired gRNAs in a PAM-in configuration frequently generated large deletions between the spaced nicks, which was not detected when using gRNAs in a PAM-out configuration with nD10A. This effect can be harnessed to boost gene-drive propagation when specifically targeting essential genes. For example, the gene-drive element can carry a DNA rescue sequence to replace a wildtype allele while restoring functionality of vital genes to ensure animal viability and gene-drive spread. If resistant alleles occur from unsuccessful allelic conversion, these mutations should produce non-viable animals that evade propagation, though small mutations in essential genes can still produce some viable escapees (Terradas et al., 2021). Therefore, large deletions induced by a gene-drive system using nH840A in a PAM-in configuration should help remove surviving escapees in a population carrying small indels.

This report also reports whether a nickase gene-drive system could reduce the formation of resistant alleles, as DNA nicks are usually repaired efficiently. Indeed, wildtype/uncut alleles were observed within the nickase experiment conditions promoting biased inheritance, and this could help reduce resistant alleles formation while facilitating further gene drive conversion. However, resistant alleles caused by single DNA nicks were also detected, which could be from non-repaired single nicks that were converted to double-strand breaks that were subsequently fixed by non-homologous end joining (Kuzminov, 2001). Importantly, mutations produced at a single target site by DNA nicks could limit further gene-drive propagation, as single nicks are poor HDR inducers (Vriend et al., 2016) and a single mutation at one target site would be enough to avoid gene-drive spread. However, the nickase system presented herein is amenable to further optimization, especially as one major contributing factor to this might be due to fixing the induced DNA paired nicks to ˜50 nucleotides apart, though in fact, efficient HDR has been observed with offset distances ranging from 20 to 100 base pairs (Vriend et al., 2016). Thus, further adapting the strategy to generate distinct offset distances to improve the HDR efficiency of the nickase-based system is recommended.

Future nickase-gene-drive approaches could explore additional optimizations to increase specificity and reduce off-target effects, which can accumulate in a population and must be considered. As two independent cleavage events need to be coordinated for the desired modification, paired nicks were shown in previous work to improve specificity while reducing off-target effects when disrupting the EMX1 gene in human cell culture(Ran et al., 2013). Recently, the off-target effects of gene drives were predicted using validated algorithms and posterior in vivo-targeted deep sequencing with Anopheles mosquitoes in laboratory cage studies (Carballar-Lejarazú et al., 2020; Garrood et al., 2021). The off-targets effects were almost undetectable if using promoters that restricted Cas9 expression to the germline. Indeed, a nickase gene-drive system could be tested in Anopheles and species with much larger genomes, such as Aedes or Culex mosquitoes(Main et al., 2021; Severson et al., 2004), to study the pervasiveness of off-target effects across genome sizes and in the wild. Furthermore, gene drives can bias Mendelian inheritance in mice (Grunwald et al., 2019; Weitzel et al.). Further, a nickase-based gene-drive system could also be applied to mice or to reduce off-target effects when editing mammalian embryos (Aryal et al., 2018).

Altogether, this Example provides the development of nickase-based gene drives that advance their applications. The use of nickase-based gene-drive systems disclosed herein is for improved population control while encouraging its implementation in a broader range of organisms.

### Summary of Example 1

CRISPR-based gene-drives have been proposed for managing insect populations, including disease-transmitting mosquitoes, due to their ability to bias their inheritance towards super-Mendelian rates (>50%). Current technologies employ a Cas9 that introduces DNA double-strand breaks into the opposing wildtype allele to replace it with a copy of the gene-drive allele via DNA homology-directed repair. Yet, the use of different Cas9 versions is unexplored, and alternative approaches could increase the available toolkit for gene-drive designs. Here, this Example reports a gene drive that relies on Cas9 nickases that generate staggered paired nicks in DNA to propagate the engineered gene-drive cassette. It shows that generating 5′ overhangs in the system yields efficient allelic conversion. The nickase gene-drive arrangement produces large, stereotyped deletions that are advantageous to eliminate viable animals carrying small mutations when targeting essential genes. This nickase gene-drive approach should expand the repertoire for gene-drive arrangements aimed at applications in mosquitoes and beyond.

### REFERENCES

Adolfi, A., Gantz, V. M., Jasinskiene, N., Lee, H.-F., Hwang, K., Terradas, G., Bulger, E. A., Ramaiah, A., Bennett, J. B., Emerson, J. J., et al. (2020). Efficient population modification gene-drive rescue system in the malaria mosquito Anopheles stephensi. Nat. Commun. 11, 5553.
Aryal, N. K., Wasylishen, A. R., and Lozano, G. (2018). CRISPR/Cas9 can mediate high-efficiency off-target mutations in mice in vivo. Cell Death Dis. 9, 1099.
Bier, E. (2021). Gene drives gaining speed. Nat. Rev. Genet.
Bothmer, A., Phadke, T., Barrera, L. A., Margulies, C. M., Lee, C. S., Buquicchio, F., Moss, S., Abdulkerim, H. S., Selleck, W., Jayaram, H., et al. (2017). Characterization of the interplay between DNA repair and CRISPR/Cas9-induced DNA lesions at an endogenous locus. Nat. Commun. 8, 13905.
Caldecott, K. W. (2008). Single-strand break repair and genetic disease. Nat. Rev. Genet. 9, 619-631.
Carballar-Lejarazu, R., Ogaugwu, C., Tushar, T., Kelsey, A., Pham, T. B., Murphy, J., Schmidt, H., Lee, Y., Lanzaro, G. C., and James, A. A. (2020). Next-generation gene drive for population modification of the malaria vector mosquito, Anopheles gambiae. Proceedings of the National Academy of Sciences 117, 22805-22814.
Chafin, D. R., Vitolo, J. M., Henricksen, L. A., Bambara, R. A., and Hayes, J. J. (2000). Human DNA ligase I efficiently seals nicks in nucleosomes. EMBO J. 19, 5492-5501.
Champer, J., Liu, J., Oh, S. Y., Reeves, R., Luthra, A., Oakes, N., Clark, A. G., and Messer, P. W. (2018). Reducing resistance allele formation in CRISPR gene drive. Proc. Natl. Acad. Sci. U.S.A 115, 5522-5527.
Champer, J., Chung, J., Lee, Y. L., Liu, C., Yang, E., Wen, Z., Clark, A. G., and Messer, P. W. (2019). Molecular safeguarding of CRISPR gene drive experiments. Elife 8.
Gantz, V. M., and Bier, E. (2015). Genome editing. The mutagenic chain reaction: a method for converting heterozygous to homozygous mutations. Science 348, 442-444.
Gantz, V. M., and Bier, E. (2016). The dawn of active genetics. Bioessays 38, 50-63.
Gantz, V. M., Jasinskiene, N., Tatarenkova, O., Fazekas, A., Macias, V. M., Bier, E., and James, A. A. (2015). Highly efficient Cas9-mediated gene drive for population modification of the malaria vector mosquito Anopheles stephensi. Proc. Natl. Acad. Sci. U.S.A 112, E6736-E6743.
Garrood, W. T., Kranjc, N., Petri, K., Kim, D. Y., Guo, J. A., Hammond, A. M., Morianou, I., Pattanayak, V., Joung, J. K., Crisanti, A., et al. (2021). Analysis of off-target effects in CRISPR-based gene drives in the human malaria mosquito. Proc. Natl. Acad. Sci. U.S.A. 118.
Gasiunas, G., Barrangou, R., Horvath, P., and Siksnys, V. (2012). Cas9-crRNA ribonucleoprotein complex mediates specific DNA cleavage for adaptive immunity in bacteria. Proc. Natl. Acad. Sci. U.S.A 109, E2579-E2586.
Gloor, G. B., Preston, C. R., Johnson-Schlitz, D. M., Nassif, N. A., Phillis, R. W., Benz, W. K., Robertson, H. M., and Engels, W. R. (1993). Type I repressors of P element mobility. Genetics 135, 81-95.
Gopalappa, R., Suresh, B., Ramakrishna, S., and Kim, H. H. (2018). Paired D10A Cas9 nickases are sometimes more efficient than individual nucleases for gene disruption. Nucleic Acids Res. 46, e71.
Grunwald, H. A., Gantz, V. M., Poplawski, G., Xu, X.-R. S., Bier, E., and Cooper, K. L. (2019). Super-Mendelian inheritance mediated by CRISPR-Cas9 in the female mouse germline. Nature 566, 105-109.
Hammond, A., Galizi, R., Kyrou, K., Simoni, A., Siniscalchi, C., Katsanos, D., Gribble, M., Baker, D., Marois, E., Russell, S., et al. (2016). A CRISPR-Cas9 gene drive system targeting female reproduction in the malaria mosquito vector Anopheles gambiae. Nat. Biotechnol. 34, 78-83.
Hammond, A. M., Kyrou, K., Bruttini, M., North, A., Galizi, R., Karlsson, X., Kranjc, N., Carpi, F. M., D′Aurizio, R., Crisanti, A., et al. (2017). The creation and selection of mutations resistant to a gene drive over multiple generations in the malaria mosquito. PLoS Genet. 13, e1007039.
Hyodo, T., Rahman, M. L., Karnan, S., Ito, T., Toyoda, A., Ota, A., Wahiduzzaman, M., Tsuzuki, S., Okada, Y., Hosokawa, Y., et al. (2020). Tandem Paired Nicking Promotes Precise Genome Editing with Scarce Interference by p53. Cell Rep. 30, 1195-1207.e7.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Kuzminov, A. (2001). Single-strand interruptions in replicating chromosomes cause double-strand breaks. Proc. Natl. Acad. Sci. U.S.A 98, 8241-8246.
Kyrou, K., Hammond, A. M., Galizi, R., Kranjc, N., Burt, A., Beaghton, A. K., Nolan, T., and Crisanti, A. (2018). A CRISPR-Cas9 gene drive targeting doublesex causes complete population suppression in caged Anopheles gambiae mosquitoes. Nat. Biotechnol. 36, 1062-1066.
Li, M., Yang, T., Kandul, N. P., Bui, M., Gamez, S., Raban, R., Bennett, J., Sánchez C, H. M., Lanzaro, G. C., Schmidt, H., et al. (2020). Development of a confinable gene drive system in the human disease vector. Elife 9.
López Del Amo, V., Leger, B. S., Cox, K. J., Gill, S., Bishop, A. L., Scanlon, G. D., Walker, J. A., Gantz, V. M., and Choudhary, A. (2020a). Small-Molecule Control of Super-Mendelian Inheritance in Gene Drives. Cell Rep. 31, 107841.
López Del Amo, V., Bishop, A. L., Sánchez C, H. M., Bennett, J. B., Feng, X., Marshall, J. M., Bier, E., and Gantz, V. M. (2020b). A transcomplementing gene drive provides a flexible platform for laboratory investigation and potential field deployment. Nat. Commun. 11, 352.
Main, B. J., Marcantonio, M., Johnston, J. S., Rasgon, J. L., Brown, C. T., and Barker, C. M. (2021). Whole-genome assembly of Culex tarsalis. G3 11.
Mali, P., Aach, J., Stranges, P. B., Esvelt, K. M., Moosburner, M., Kosuri, S., Yang, L., and Church, G. M. (2013). CAS9 transcriptional activators for target specificity screening and paired nickases for cooperative genome engineering. Nat. Biotechnol. 31, 833-838.
Port, F., Chen, H.-M., Lee, T., and Bullock, S. L. (2014). Optimized CRISPR/Cas tools for efficient germline and somatic genome engineering in Drosophila. Proc. Natl. Acad. Sci. U.S.A 111, E2967-E2976.
Ran, F. A., Hsu, P. D., Lin, C.-Y., Gootenberg, J. S., Konermann, S., Trevino, A. E., Scott, D. A., Inoue, A., Matoba, S., Zhang, Y., et al. (2013). Double nicking by RNA-guided CRISPR Cas9 for enhanced genome editing specificity. Cell 154, 1380-1389.
Reyes, G. X., Kolodziejczak, A., Devakumar, L. J. P. S., Kubota, T., Kolodner, R. D., Putnam, C. D., and Hombauer, H. (2021). Ligation of newly replicated DNA controls the timing of DNA mismatch repair. Curr. Biol. 31, 1268-1276.e6.
Severson, D. W., DeBruyn, B., Lovin, D. D., Brown, S. E., Knudson, D. L., and Morlais, I. (2004). Comparative genome analysis of the yellow fever mosquito Aedes aegypti with Drosophila melanogaster and the malaria vector mosquito Anopheles gambiae. J. Hered. 95, 103-113.
Simoni, A., Hammond, A. M., Beaghton, A. K., Galizi, R., Taxiarchi, C., Kyrou, K., Meacci, D., Gribble, M., Morselli, G., Burt, A., et al. (2020). A male-biased sex-distorter gene drive for the human malaria vector Anopheles gambiae. Nat. Biotechnol. 38, 1054-1060.
Terradas, G., Buchman, A. B., Bennett, J. B., Shriner, I., Marshall, J. M., Akbari, O. S., and Bier, E. (2021). Inherently confinable split-drive systems in Drosophila. Nat. Commun. 12, 1480.
Vriend, L. E. M., and Krawczyk, P. M. (2017). Nick-initiated homologous recombination: Protecting the genome, one strand at a time. DNA Repair 50, 1-13.
Vriend, L. E. M., Prakash, R., Chen, C.-C., Vanoli, F., Cavallo, F., Zhang, Y., Jasin, M., and Krawczyk, P. M. (2016). Distinct genetic control of homologous recombination repair of Cas9-induced double-strand breaks, nicks and paired nicks. Nucleic Acids Res. 44, 5204-5217.
Wang, H., and Hays, J. B. (2007). Human DNA mismatch repair: coupling of mismatch recognition to strand-specific excision. Nucleic Acids Res. 35, 6727-6739.
Wang, Q., Liu, J., Janssen, J. M., Le Bouteiller, M., Frock, R. L., and Gonçalves, M. A. F. V. (2021). Precise and broad scope genome editing based on high-specificity Cas9 nickases. Nucleic Acids Res. 49, 1173-1198.
Wang, Y., Zhao, J., Duan, N., Liu, W., Zhang, Y., Zhou, M., Hu, Z., Feng, M., Liu, X., Wu, L., et al. (2018). Paired CRISPR/Cas9 Nickases Mediate Efficient Site-Specific Integration of F9 into rDNA Locus of Mouse ESCs. International Journal of Molecular Sciences 19, 3035.
Weitzel, A. J., Grunwald, H. A., Levina, R., Gantz, V. M., Hedrick, S. M., Bier, E., and Cooper, K. L. Meiotic Cas9 expression mediates genotype conversion in the male and female mouse germline.
Xu, X.-R. S., Gantz, V. M., Siomava, N., and Bier, E. (2017). CRISPR/Cas9 and active genetics-based trans-species replacement of the endogenous -L2 CRM reveals unexpected complexity. Elife 6.

### Example 2

### Development of Gene Drive Technologies for the Suppression of Culex Mosquitos

### Introduction

The Culex pipiens species complex includes several species and their hybrid forms, including Cx. pipiens pipiens, Cx. pipiens pallens, Cx. pipiens molestus, Cx. quinquefasciatus, Cx. australicus, and Cx. globocoxitus. While some of these species have limited distribution, Cx. pipiens pipiens, Cx. quinquefasciatus, and their hybrids are widespread globally and represent the primary urban and suburban disease vector [1]. The ability of these species to produce fertile hybrids has been a source of an ongoing discussion around the taxonomy of these species [2] and has been proposed as a mechanism explaining the surprising adaptability of these mosquitoes such as the “molestus” form of Cx. pipiens adapted to survive in the underground network of London [3] and may account for the rapid evolution of insecticide resistance across the Culex pipiens complex [4,5].

These mosquitoes are vectors for several medically-relevant RNA viruses such as West Nile virus, Japanese Encephalitis virus, Rift Valley fever virus, Western Equine Encephalitis virus, Eastern Equine Encephalitis virus, Sindbis virus, and St. Louis Encephalitis virus, as well as nematodes causing lymphatic filariasis [1]. Although fatalities in the United States due to these diseases are usually low in number, Culex mosquitoes are a permanent disease vector that represents a fertile ground for the establishment of new pathogens. For example, it has been estimated that since its introduction in the United States in 1999, West Nile Virus hospitalizations alone amounted on average to ˜$56M per year [6]. The mixed feeding behavior of these mosquitos on birds and mammals creates seasonal cycles of pathogen amplification in wild animals, which can lead to a periodic increase in the risk of pathogen transmission to people and to unpredictable outbreaks [7]. Lastly, the zoonotic nature of these and other viruses allows for unchecked reservoirs of viruses, mostly in wild birds, that could act as evolutionary melting pots for the development of new viral forms: the Western Equine Encephalitis is considered to be the outcome of recombination between EEEV and a Sindbis-like virus [8]. Because within this species complex, Culex quinquefasciatus (Cx. quin.) is considered the disease vector with the greatest human health impact worldwide [9], and its genetic adaptability has made it a challenging vector to keep at bay, therefore, this species were chosen for studies presented in this Example.

Genetic technologies such as engineered gene drives (GDs) based on homing endonucleases were proposed more than a decade ago [10]. Recently, CRISPR has allowed the development of these and other technologies that use genetically-engineered vectors to modify or suppress wild populations [11,12]. While different strategies have been proposed for their application, GDs were successfully applied to various organisms including yeast [13], fruit flies [14,15], Anopheles [16-18] and Aedes [19] mosquitoes, and the mouse [20].

Recently, tools for the expression of CRISPR components have been described in Aedes [19]. Evaluation of CRISPR activity in this species has been reported by few groups 15 [27-30], but no extensive CRISPR technology application has been achieved, mostly due to the fewer tools available for this species. The advent of CRISPR has caused the development of a flurry of new scientific opportunities and technologies. However, transgenesis and genome editing in mosquitoes remain cumbersome and labor-intensive. Compared to Anopheles and Aedes, Culex has been somewhat neglected regarding the development of genetic suppression strategies. For Anopheles mosquitoes, these techniques have proceeded swiftly due to the vast body of knowledge available due to the longstanding interest in this vector and their Plasmodium parasite causing malaria. Aedes and Culex mosquitoes have been somewhat lagging on CRISPR technology development [31]. Only few studies reported transgenesis in Culex [32,33], with limited follow-up use in the field.

### Descriptions & Results

The studies presented in this Example close this gap by jumpstarting CRISPR-based technologies in Cx. quin. and/or provide tools and knowledge for the genetic engineering of this species and develop a GD system for the population suppression of this vector. This Example provides data, molecular tools, and genome-editing know-how that would constitute the bedrock to develop field-ready products. More specifically, this Example provides the building blocks for the expression of CRISPR components, the development of the know-how for site-specific transgenesis, and a GD built for the suppression of Cx. quin. populations. The implementation of suppression GD provided herein would constitute an additional weapon in the arsenal to suppress vector populations and disease outbreaks.

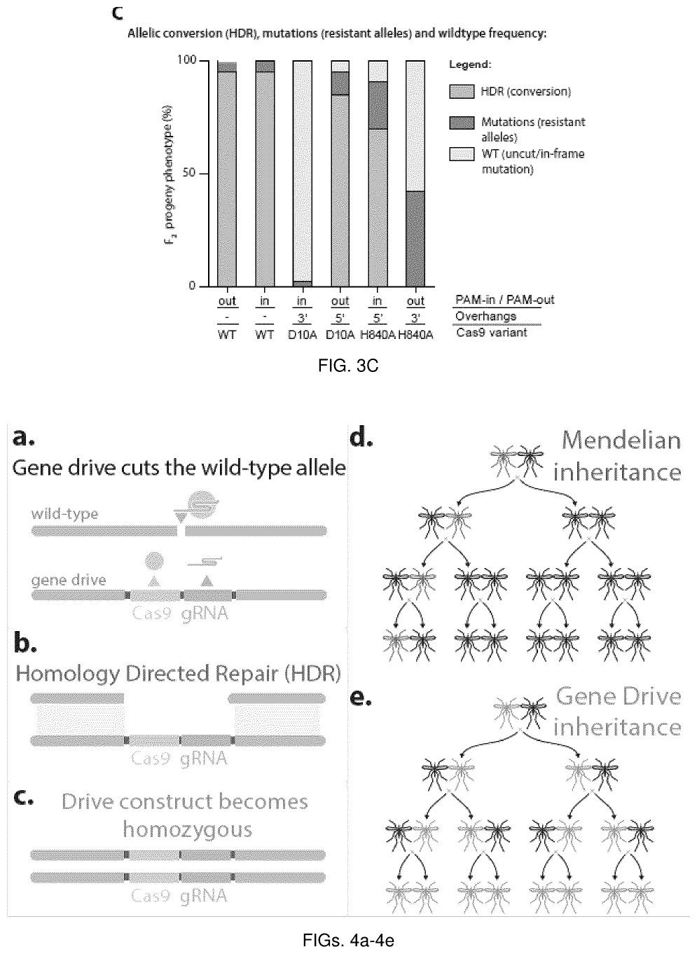

GD systems promote the propagation of the drive elements along with engineered traits to fight pathogens or, alternatively, deleterious ones to suppress the population itself. The drive process involves cutting the wild-type chromosome and repairing it using the GD allele as a template (FIGS. 4a-4e). While this process is remarkably efficient, a failure of copying results in the generation of small indels (insertions/deletions). These indel alleles disrupt the gRNA target site and are therefore resistant to further Cas9 cutting, remaining in the target population, and effectively working against the spread of a GD. Insertion of CRISPR GDs within essential genes along with a fragment rescuing the gene function would result in eventual indels disrupting the essential gene and having a high fitness cost compared to the GD, and their removal from the population by negative selection, therefore ensuring efficient GD spread [12]. Since the genetic engineering of Culex mosquitoes is in its infancy, this Example describes the development of a suppression GD which does not require the development of effector genes in addition to the GD construct.

Two development stages are described in this Example, each of which is tackling each a crucial aspect of technology development. The first development is to build and validate constructs with promoter sequences suitable to drive the expression of CRISPR components in Cx. quin. which is a necessary step for the development of any genetic technology. The second development is to develop targeted transgenesis of a ubiquitous Cas9 line to facilitate future engineering of this species and a GD construct for population suppression.

A. Develop Elements for the Expression of Cas9 and gRNA in Culex quinquefasciatus

Two laboratories have recently reported the use of CRISPR components (Cas9 protein and gRNA) injected in Cx. quin. eggs to generate heritable mutations [27,28]. To support the feasibility these methods were conducted in the laboratory Cx. quin. colony (originally isolated in the 1950s from California), and for which a high-quality genome assembly was generated. The findings from the exploratory reports were replicated and expanded by targeting several recessive pigmentation genes: white (CPIJ005542), kynurenine hydroxylase (CPIJ017147), cardinal (CPIJ005949), yellow (CPIJ018481), and ebony (CPIJ003423). This work, now available as a preprint on BiorXiv [30], demonstrates the ability to work with and perform research on Culex mosquitoes, provides with an efficient pipeline to select and test gRNAs, and further supports the feasibility of the work described in this Example.

To develop GD technologies in this species, first, promoters' sequences are identified and tested that can be used to efficiently drive the expression of CRISPR components. Four Cas9 plasmids and four gRNA ones were built and tested, and their functions in vivo were evaluated.

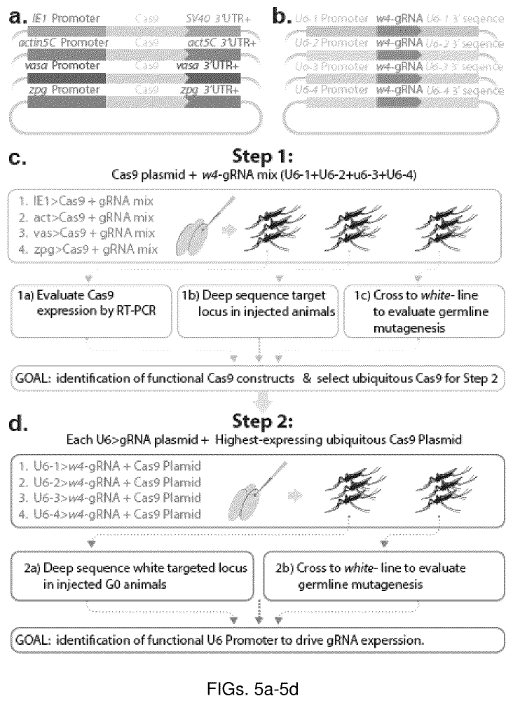

More specifically, two ubiquitous promoters were chosen to drive the expression of Cas9: the first is a strong viral promoter commonly used for expression in insect systems, IE1 [35], and the second is an endogenous ubiquitously-expressed gene Actin5C (CPIJ009808), which was successfully used in Drosophila [36]. IE1 was chosen because it was tested in Cx. quin. and its ability to drive GFP was validated (FIG. 5d). The choice of Actin5C is driven by the fact that high levels of Cas9 have been shown to be toxic in different systems, including in Anopheles [16]. Therefore, as strong viral promoters, such as IE1, could lead to toxicity. To avoid this potential issue, this endogenous promoter was used. Separately, two germline-specific promoters that could be and/or have been successfully used in a GD strategy are also selected. The first promoter is the vasa (vas, CPIJ009286) gene that has been successfully used to generate GD in Drosophila melanogaster [16,21,37], Anopheles stephensi [16], and the mouse [20]. The second promoter is from zero-population-growth (zpg, CPIJ000259) that has been used in suppression gene-drive strategy in Anopheles gambiae [17]. For Actin5C, vasa, and zpg, the endogenous sequences from the wild-type line are cloned and fragmented to ensure functionality in the system.

For the expression of gRNAs, the use of Pol-III promoters driving U6-small-nuclear-RNAs is focused on, which have been shown to be a reliable method to drive the expression of gRNAs in multiple systems ranging from human [38], to plants [39], to insects [16,40]. Seven U6 genes were identified in the Cx. quin. genome U6-1—CPIJ039653, U6-2—CPIJ039728, U6-3—CPIJ039543, U6-4—CPIJ039801, U6-5—CPIJ040819, U6-6—CPIJ039596, U6-7—CPIJ040693), some of which were previously tested in Cx. quin. cells for activity [41]. Four of them (U6-1, -2, -3, -4) were chosen for testing because they displayed the highest transcript sequence conservation to other Diptera, making them less likely to be pseudogenes or tissue-specific splicing factors. For each promoter, a gRNA sequence targeting the white gene of Cx. quin was cloned. The the w4-gRNA was chosen because it was previously tested and displayed high activity, and for which the white-mosaic or mutant phenotypes are easily scored and do not display any viability issues [30].

For each of the eight promoters chosen to drive either Cas9 and gRNAs, their sequences are cloned in front of a Cas9 gene optimized for Anopheles [16] or the w4-gRNA which displayed the highest activity in the previous analysis targeting the white gene [30], respectively. For the Cas9 constructs are selected about ˜4 kb upstream of the gene's start codon. a fragment of the gene downstream of the stop codon, including the 3′UTR sequence and an additional ˜500 bp is also cloned, which cover all the sequences necessary to maintain endogenous regulation of the mRNA termination. For the Cas9 construct using the IE1 promoter, the SV40 poly-A sequence is used. For each of the constructs for the expression of the w4-gRNA, the gRNA sequence in between ˜700 bp of the promoter and ˜200 bp of terminator sequences are cloned; while these constructs could be smaller, additional sequences on either side to maintain some of the genomic sequences surrounding the genes may also be chosen. These constructs, outlined in FIGS. 5a-5b are tested in a stepwise fashion to evaluate their functionality, and the best candidates for the generation of transgenic animals are identified.

More specifically, in the first step, four injections are performed, each of them having one of the four Cas9 constructs, together with a mix of the four gRNA constructs. 500 ug/uL of a 4:1:1:1:1 ratio of each Cas9:U6-1:U6-2:U6-3:U6-4 constructs are injected into ˜600 Cx. quin. embryos, and the DNA concentration is lower if toxic. ˜60-90 surviving adults are expected for each injection based on the survival rate (10-15%) obtained in the previous analysis [30]. The surviving animals are then inspected for the presence of the expected white-eye phenotype (either fully-penetrant or in mosaic form), which indicate both Cas9 and gRNA activity. While phenotypes for the ubiquitous Cas9 promoters we are expected, it is not clear whether the germline ones would lead to visible genome edits in the soma of in the injected G0 animals. Therefore, with the surviving animals of each injection, the following analyses are performed:
1a) To evaluate Cas9 expression (FIG. 5c), for each of the four injections 10-15 males and females are picked and squished together to prepare RNA. A reverse transcriptase (RT)-PCR reaction is performed using Cas9-specific primers, to amplify a segment of the Cas9 gene and its presence is detected. Uninjected wild-type animals are used as the negative control. This method is sensitive enough to detect even the lower levels of expression driven by the germline promoters.
1b) To evaluate gRNA activity (FIG. 5d), an additional 10-15 males and females are selected and genomic DNA (animals also squished together) are prepared. These four DNA isolates are subjected to a PCR-amplicon deep-sequencing of the w4-gRNA-targeted region. This technique generates ˜50,000 reads of the white locus and is able to detect cutting at the target site with extreme sensitivity. As a positive control, a smaller injection of Cas9 protein and w4-gRNA is perform, respectively, which is known to effectively cleaves the white locus, as shown in the previous study [30]. Some level of cutting detected as small indels (insertion/deletions) at the cut site in a subset of the reads are expected, indicating both Cas9 and gRNA activity for that specific injection condition.
1c) To evaluate both Cas9 and gRNA activity in yet another parallel way, for each injection, the remaining 40-60 adult mosquitoes are mated as separate males and females pools to a homozygous white-mutant line in the laboratory. Their G1 progeny is screened for the presence of the expected white-eye phenotype (as done in previous analysis [30]); any white-eye G1 animal indicates a successful germline editing event, further supporting the activity of the injected plasmids.

The germline constructs might be expressed at very low levels, especially when present as plasmids, and could lead to failure in 1a), 1b) or 1c). For negative results in 1a) a nested PCR is performed to increase detection. For 1b) additional injection (˜200 eggs) with higher plasmid concentrations is performed. If 1c) fails, the results in 1a) and 1b) are relied on to bring the analyses forward to the second step.

Thus, altogether, the above analyses identify which of the Cas9 constructs is expressed, and their expression levels relative to each other (although ubiquitous and germline drivers are hard to compare). In addition, cutting activity detected in either 1 b) or 1c) indicate that at least one of the U6 promoters fused to the w4-gRNA can drive gRNA expression. The G0 injected adults from 1c) after mating are frozen and stored for additional replicates of the analysis in 1a) or 1 b), if needed.

The second step focuses on identifying U6 promoters with high activity. Here, a similar injection is performed, as described above, to test in each of four injections a single gRNA construct in combination with the ubiquitous-Cas9 element that provides the highest editing rate in the first step analyses discussed above. ˜400 Cx. quin. embryos are injected with 500 ug/uL (or lower, depending on the previous results) of a 1:1 ratio of the chosen Cas9:U6 construct. The surviving animals are first inspected for the presence of the expected white-eye phenotype. Even if not present in the previous analyses, a higher functional gRNA concentration injected here, may lead to at least a mosaic phenotype. With the surviving animals (˜40-60) the following analyses are performed:
2a) Similar to 1 b) above, to evaluate the activity of each gRNA promoter, ˜10-15 males and females are selected, and genomic DNA (animals squished together) is prepared. The four DNA isolates are subjected to the same PCR-amplicon deep-sequencing of the w4-gRNA-targeted region described above. The ˜50,000 reads generated for each of the U6 promoters are evaluated and any detected cutting at the targeted locus is compared. As a positive control, one of the positive samples prepared in 1b) is use. Some level of cutting detected as small indels in each injection condition is expected, and the percentage of edited sequences in each injection is compared to identify the U6 promoters displaying the highest activity.
2b) Similar to 1c) above, in parallel, the remaining 30-45 adult male and female mosquitoes in two pools are mated to the white-mutant line, and their G1 progeny is screened for the presence of the expected white-eye phenotype. While scoring the G1 offspring is a semi-quantitative measure of U6 expression, together with the data observed in 2a) provide an idea of relative activity.

Thus, this second step identifies one or more U6-Promoter constructs that can sustain efficient expression of the gRNA in Cx. quin. As for the analyses in first step, the G0 injected adults from 2b) after mating are stored to generate additional replicates of the analysis in 2a), if needed.

In summary, through the analyses of the two steps discussed above for the first development stage, expression information and functional validation of the built constructs are obtained, and three (3) candidates are identified: one ubiquitous-Cas9, one germline-Cas9, and one U6-gRNA construct. In certain cases, three additional U6 genes are kept as a backup in case the first four fail. Alternatively, other Pol-III promoters such as H1 or 7SK [41,42] are also tested.

B. Develop HDR-Based Transgenesis and GD in Culex quinquefasciatus

To develop CRISPR GD strategies, engineered transgenes need to be delivered precisely at predetermined locations in the genome, and therefore transposon-based integration would not be a viable option. Therefore, it is essential to develop targeted HDR-based delivery of transgenic elements in Culex mosquitoes. This development stage focused on testing and building the know-how for site-specific transgenesis. While doing so, animals carrying two transgenic constructs are generated and used for the work including but not limited to, 1) a ubiquitous-Cas9, and 2) a GD for the suppression of Cx. quin. populations.

The choice of the first transgene stands in the value of a ubiquitous-Cas9-expressing Cx. quin. line that could be used as a platform for targeted-mutagenesis which is higher in animals expressing Cas9 endogenously. Additionally, it could function as a tool for high-efficiency genome editing of endogenous candidates relevant to the pathogenesis of disease agents by injecting gRNAs. The transgene producing the highest editing in the analyses of the first development stage is used, and to mitigate risk, its insertion is targeted into the white locus, which is healthy as a homozygous line. The w4-gRNA is used. which provides consistent high-editing rates [30].

The second construct is a GD element for population suppression. This construct is much bigger in size, as it includes a Cas9, a gRNA, and a marker gene, potentially rendering it harder to insert in the genome. Its insertion is targeted in the doublesex (dsx) locus, which has been successfully used in Anopheles in a GD strategy [17]. Briefly, dsx is a gene with two splicing isoforms dsxM(male) and dsxF (female) that are expressed in the two sexes to determine sexual dimorphism in dipterans [43]. Previous work has used a GD to disrupt the dsxF splicing acceptor leading to the splicing forced to the dsxM acceptor and therefore generating only male progeny [17]. This strategy could be prone to the generation of small indels, at the targeted site, that are resistant to further attack by Cas9 and yet maintain the correct splicing of the dsxF isoform. Indels were not observed in this work [17], but could be problematic if released in the wild.

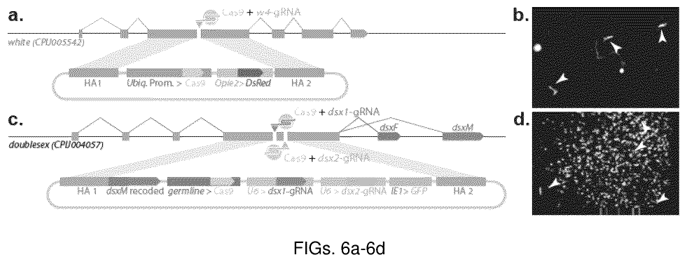

Differently from the previous work, one of the dsx exons common to both sexes' isoforms is targeted, and a re-coded version of the dsxM transcript is provided. Additionally, two closely-located (˜25 bp) highly-efficient gRNAs used in a Protospacer Adjacent Motif (PAM) are used—in arrangement that the previous work [21] predicts to be the best situation to lead to efficient HDR-conversion when using multiplexing (FIG. 6b).

The design of this GD improves differs from the previous work as it uses multiplexing with two gRNAs in an arrangement that would allow each one of them to fail and still lead to conversion. Additionally, resistant alleles maintaining the gene function are even less likely since the coding sequence is now targeted by two gRNAs. Even in such an unlikely situation, the GD always forces the sex determination towards the male sex. Altogether the features of this design should lead to an efficient suppression gene drive.

Generation of a Cx. quin. Cas9 line: the construct is generated as outlined in FIG. 6a by cloning two ˜1 kbp homology arm sequences abutting each side of the w4-gRNA cut site on the white gene. These sequences are cloned on each side of a central cassette containing one of the two ubiquitous Cas9 constructs tested in the first development stage (the construct with the highest expression is selected), and a DsRed-expressing marker is cloned under the control of the viral promoter Opie2 [44]. This construct was tested by injecting it in Cx quin., eggs and its transient expression was observed (FIG. 6b).

Next, batches of ˜800 eggs with a mix containing the generated HDR template plasmid, Cas9 protein, and synthesized w4-gRNA are injected, following the protocol similar to what have been optimized in the previous work [30]. The resulting G0 injected adults are crossed in small batches of ˜5 G0 males to 20 wild-type females or ˜10 G0 females to 15 wild-type males. Following blood feeding, the G1 offspring is screened for the presence of the fluorescent marker. The blood-feeding is repeated to induce egg-production 2 or 3 consecutive times for the same G0 batch before proceeding to subsequent rounds of injections. Based on transgenesis in other mosquito species, with a rate of ˜1-2% [16,45] and the survival rate to adulthood of ˜10% [30], transformants are expected to obtain by performing ˜3 rounds of injections.

Positive transformants are single-crossed to wild-type animals of the opposite sex, and after obtainment of the following generation, and verified that the fluorescent transgene is observed in such progeny, each G1 transgenic parent is killed for molecular characterization of the insertion. PCR amplification of the junction points is first performed, employing a primer located on the genome and outside of each homology arm, and one within the transgenic sequence. Positive confirmation of the transgene junction points are followed by PCR amplification of all or sections of the transgene, to verify correct insertion of all its sequence.

Generation of a Cx. quin. gene-drive line: The first step to generate the GD construct is to validate the gRNAs to be used, and given that this strategy uses two gRNAs, two couples of gRNAs (PAM-in, and with cuts at ˜25 bp distance) are selected. The efficiency of these gRNAs are then tested by injecting each one of the selected four, in combination with Cas9 protein into ˜200 eggs. The injected eggs are let to develop for 24 h and then used to prepare genomic DNA. The DNA is then subject to deep-sequencing of a PCR amplicon covering the targeted sequence (similar to 1b) in the first development stage). Analysis of the ˜50.000 sequences obtained yields confirmation of activity and an estimate of the efficiency of the selected gRNAs, and which selected couple are used to generate the GD construct.

Two homology arms are then cloned by PCR-amplifying the sequences abutting to the cut site of either the selected gRNAs (FIG. 6c). In between them, it is inserted: a) a recoded version of the dsxM transcript, b) the selected germline promoter (chosen based on the analyses in the first development stage), c) two genes expressing the selected gRNAs (dsx1 and dsx2) under the control of functional U6 promoters, and d) a visible marker including an IE1 promoter driving the expression of GFP (FIG. 6d), previously tested along with the Opie2>DsRed (FIG. 6c). The resulting plasmid (FIG. 6c) is inserted in the dsx locus by co-injecting it with Cas9 protein and synthesized dsx1- and dsx2-gRNAs. The G0 animals are crossed to wild-type, following a protocol similar to what was described above for the Cas9 line. Transformants are first identified by screening for the presence of GFP signal (FIG. 6d), and then their correct transgene insertion is determined by stepwise PCR amplification of the junction sites and then the rest of the inserted transgene.

From this development stage, two Cx. quin. Lines are obtained: 1) a Cas9-expressing line that boosts genome editing and transgenesis, and 2) a gene-drive line that is the basis for a larger project focusing on assessing its ability to suppress laboratory populations.

Transgenesis in mosquitos has well-known low efficiencies, and the previous experience with inserting a GD construct in Anopheles stephensi yielded 2 transformants out of ˜25,000 F1 screened individuals [16]. This could result in the failure to isolate transformants in the second development stage. In the Cas9-line injection, if no transformants are observed after the first round of injection, a similar injection is first performed and the G0 injected animals are crossed to the white-line instead, to evaluate if cutting is happening at all, or at lower levels; and then the protocol is adjusted accordingly by increasing or changing concentrations of the injected mix. An alternative solution to this issue is to generate Cas9-expressing mosquito lines using traditional transgenesis which was shown to work in Cx. quin. using a Hermes transposon [33]. While HDR-based transgenesis is focused here, this could be a backup strategy for the generation of the ubiquitous-Cas9 line, as the same construct that is built could be readily cloned into a Hermes backbone. Such a line could increase the efficiency of Cas9 edits as observed and reported [46] that could be instrumental in achieving HDR-based site-directed transgenesis.

If major issues with the generation of transformants are encountered, the generation of the Cas9 line is then focused on, as this could be used to later obtain the GD insertion at higher frequencies. For the GD construct, two potential issues are anticipated: 1) altered sex-related morphologies and potential sterility, which could be an issue in recovering transformants as injected animals might be infertile or unable to mate. In such a case, the concentration of the injected mix is first lowered to lower the effect. If this does not work, only the plasmid is injected with, as the Cas9 gene on the GD construct should be expressed only in the germline and resulting in editing only in that tissue, which should avoid any pleiotropic effects that injecting with Cas9 protein might have. 2) the second point regards the GD transformants, which are males if the GD works as predicted. To keep this line the GD males need to be crossed to wild-type females at each generation. This process is also a preliminary test of the GD as the progeny of such crosses should have a skew towards males.

The functionality of the generated Cas9 line is validated. To do so, this GD line is injected with a plasmid expressing a gRNA targeting another previously described pigmentation marker, yellow (CPIJ018481), and the Cas9 activity of the generated line is evaluated by analyzing the yellow phenotype in the G0 injected animals and their G1 offspring. Such a validated reagent is a valuable tool for genome engineering of this species using CRISPR. Further, the ability of this GD line to copy on the companion chromosome and resulting in GD offspring to be >50% males is tested. And a follow up step is to analyze how this GD is able to spread into and suppress the laboratory caged populations of the wild-type strain, as well as other populations with different genetic makeup.

### REFERENCES

1. Farajollahi A, Fonseca D M, Kramer L D, Marm Kilpatrick A. “Bird biting” mosquitoes and human disease: a review of the role of Culex pipiens complex mosquitoes in epidemiology. Infect Genet Evol. 2011; 11: 1577-1585.
2. Harbach R E. Culex pipiens: Species Versus Species Complex—Taxonomic History and Perspective. Journal of the American Mosquito Control Association. 2012. pp. 10-23. doi:10.2987/8756-971x-28.4.10
3. Byrne K, Nichols R A. Culex pipiens in London Underground tunnels: differentiation between surface and subterranean populations. Heredity. 1999; 82 (Pt 1): 7-15.
4. Liu H, Cupp E W, Micher K M, Guo A, Liu N. Insecticide Resistance and Cross-Resistance in Alabama and Florida Strains of Culex quinquefaciatus. Journal of Medical Entomology. 2004. pp. 408-413. doi:10.1603/0022-2585-41.3.408
5. Kothera L, Nelms B M, Reisen W K, Savage H M. Population genetic and admixture analyses of Culex pipiens complex (Diptera: Culicidae) populations in California, United States. Am J Trop Med Hyg. 2013; 89: 1154-1167.
6. Staples J E, Shankar M B, Sejvar J J, Meltzer M I, Fischer M. Initial and long-term costs of patients hospitalized with West Nile virus disease. Am J Trop Med Hyg. 2014; 90: 402-409.
7. Turell M J, Sardelis M R, Dohm D J, O'Guinn M L. Potential North American vectors of West Nile virus. Ann N Y Acad Sci. 2001; 951: 317-324.
8. Hahn C S, Lustig S, Strauss E G, Strauss J H. Western equine encephalitis virus is a recombinant virus. Proc Natl Acad Sci USA. 1988; 85: 5997-6001.
9. Samy A M, Elaagip A H, Kenawy M A, Ayres C F J, Peterson A T, Soliman D E. Climate Change Influences on the Global Potential Distribution of the Mosquito Culex quinquefasciatus, Vector of West Nile Virus and Lymphatic Filariasis. PLoS One. 2016; 11: e0163863.
10. Burt A. Site-specific selfish genes as tools for the control and genetic engineering of natural populations. Proc Biol Sci. 2003; 270: 921-928.
11. Gantz V M, Akbari O S. Gene editing technologies and applications for insects. Curr Opin Insect Sci. 2018; 28: 66-72.
12. Esvelt K M, Smidler A L, Catteruccia F, Church G M. Emerging technology: concerning RNA-guided gene drives for the alteration of wild populations. Elife. 2014; 3: e03401.
13. DiCarlo J E, Chavez A, Dietz S L, Esvelt K M, Church G M. Safeguarding CRISPR-Cas9 gene drives in yeast. Nat Biotechnol. 2015; 33: 1250-1255.
14. Gantz V M, Bier E. Genome editing. The mutagenic chain reaction: a method for converting heterozygous to homozygous mutations. Science. 2015; 348: 442-444.
15. Kandul N P, Liu J, Sanchez C H M, Wu S L, Marshall J M, Akbari O S. Transforming insect population control with precision guided sterile males with demonstration in flies. Nat Commun. 2019; 10: 84.
16. Gantz V M, Jasinskiene N, Tatarenkova O, Fazekas A, Macias V M, Bier E, et al. Highly efficient Cas9-mediated gene drive for population modification of the malaria vector mosquito Anopheles stephensi. Proc Natl Acad Sci USA. 2015; 112: E6736-43.
17. Kyrou K, Hammond A M, Galizi R, Kranjc N, Burt A, Beaghton A K, et al. A CRISPR-Cas9 gene drive targeting doublesex causes complete population suppression in caged Anopheles gambiae mosquitoes. Nat Biotechnol. 2018; 36: 1062-1066.
18. Hammond A, Galizi R, Kyrou K, Simoni A, Siniscalchi C, Katsanos D, et al. A CRISPR-Cas9 gene drive system targeting female reproduction in the malaria mosquito vector Anopheles gambiae. Nat Biotechnol. 2016; 34: 78-83.
19. Li M, Bui M, Yang T, Bowman C S, White B J, Akbari O S. Germline Cas9 expression yields highly efficient genome engineering in a major worldwide disease vector, Aedes aegypti. Proc Natl Acad Sci USA. 2017; 114: E10540-E10549.
20. Grunwald H A, Gantz V M, Poplawski G, Xu X-RS, Bier E, Cooper K L. Super-Mendelian inheritance mediated by CRISPR-Cas9 in the female mouse germline. Nature. 2019; 566: 105-109.
21. López Del Amo V, Bishop A L, SAnchez C. HM, Bennett J B, Feng X, Marshall J M, et al. A transcomplementing gene drive provides a flexible platform for laboratory investigation and potential field deployment. Nat Commun. 2020; 11: 352.
22. Del Amo V L, Leger B S, Cox K J, Gill S, Bishop A L, Scanlon G D, et al. Small-molecule control of super-Mendelian inheritance in gene drives. bioRxiv. 2019. p. 665620. doi:10.1101/665620
23. Guichard A, Haque T, Bobik M, Xu X-RS, Klanseck C, Kushwah R B S, et al. Efficient allelic-drive in Drosophila. Nat Commun. 2019; 10: 1640.
24. Akbari O S, Bellen H J, Bier E, Bullock S L, Burt A, Church G M, et al. BIOSAFETY. Safeguarding gene drive experiments in the laboratory. Science. 2015; 349: 927-929.
25. Adelman Z, Akbari O, Bauer J, Bier E, Bloss C, Carter S R, et al. Rules of the road for insect gene drive research and testing. Nat Biotechnol. 2017; 35: 716-718.
26. National Academies of Sciences, Engineering, and Medicine, Division on Earth and Life Studies, Board on Life Sciences, Committee on Gene Drive Research in Non-Human Organisms: Recommendations for Responsible Conduct. Gene Drives on the Horizon: Advancing Science, Navigating Uncertainty, and Aligning Research with Public Values. National Academies Press; 2016.
27. Anderson M E, Mavica J, Shackleford L, Flis I, Fochler S, Basu S, et al. CRISPR/Cas9 gene editing in the West Nile Virus vector, Culex quinquefasciatus Say. PLoS One. 2019; 14: e0224857.
28. Li M, Li T, Liu N, Raban R R, Wang X, Akbari O S. Methods for the generation of heritable germline mutations in the disease vector Culex quinquefasciatus using clustered regularly interspaced short palindrome repeats-associated protein 9. Insect Molecular Biology. 2019. doi:10.1111/imb.12626
29. Itokawa K, Komagata O, Kasai S, Ogawa K, Tomita T. Testing the causality between CYP9M10 and pyrethroid resistance using the TALEN and CRISPR/Cas9 technologies. Scientific Reports. 2016. doi:10.1038/srep24652
30. Feng X, Kambic L, Nishimoto J H K, Reed F A, Denton J A, Sutton J T, et al. Evaluation of pigmentation gene knock-outs by CRISPR as potential targets for the genetic engineering of Culex quinquefasciatus. Cold Spring Harbor Laboratory. 2020. p. 2020.10.21.349704. doi:10.1101/2020.10.21.349704
31. Wilke A B B, Beier J C, Benelli G. Transgenic Mosquitoes—Fact or Fiction? Trends in Parasitology. 2018. pp. 456-465. doi:10.1016/j.pt.2018.02.003
32. Allen M L, Christensen B M. Flight muscle-specific expression of act88F: GFP in transgenic Culex quinquefasciatus Say (Diptera: Culicidae). Parasitol Int. 2004; 53: 307-314.
33. Allen M L, O'Brochta D A, Atkinson P W, Levesque C S. Stable, germ-line transformation of Culex quinquefasciatus (Diptera: Culicidae). J Med Entomol. 2001; 38: 701-710.
34. Adolfi A, Gantz V M, Jasinskiene N, Lee H-F, Hwang K, Bulger E A, et al. Efficient population modification gene-drive rescue system in the malaria mosquito Anopheles stephensi. 2020. p. 2020.08.02.233056. doi:10.1101/2020.08.02.233056
35. Guarino L A, Summers M D. Nucleotide sequence and temporal expression of a baculovirus regulatory gene. J Virol. 1987; 61: 2091-2099.
36. Port F, Chen H-M, Lee T, Bullock S L. Optimized CRISPR/Cas tools for efficient germline and somatic genome engineering in Drosophila. Proc Natl Acad Sci USA. 2014; 111: E2967-76.
37. Champer J, Reeves R, Oh S Y, Liu C, Liu J, Clark A G, et al. Novel CRISPR/Cas9 gene drive constructs reveal insights into mechanisms of resistance allele formation and drive efficiency in genetically diverse populations. PLoS Genet. 2017; 13: e1006796.
38. Cong L, Ran F A, Cox D, Lin S, Barretto R, Habib N, et al. Multiplex genome engineering using CRISPR/Cas systems. Science. 2013; 339: 819-823.
39. Xie K, Yang Y. RNA-guided genome editing in plants using a CRISPR-Cas system. Mol Plant. 2013.
40. Gratz S J, Cummings A M, Nguyen J N, Hamm D C, Donohue L K, Harrison M M, et al. Genome engineering of Drosophila with the CRISPR RNA-guided Cas9 nuclease. Genetics. 2013; 194: 1029-1035.
41. Anderson M A E, Purcell J, Verkuijl S A N, Norman V C, Leftwich P T, Harvey-Samuel T, et al. Expanding the CRISPR Toolbox in Culicine Mosquitoes: In Vitro Validation of Pol Ill Promoters. ACS Synth Biol. 2020; 9: 678-681.
42. Gao Z, Herrera-Carrillo E, Berkhout B. A Single H1 Promoter Can Drive Both Guide RNA and Endonuclease Expression in the CRISPR-Cas9 System. Mol Ther Nucleic Acids. 2019; 14: 32-40.
43. Lutrat C, Giesbrecht D, Marois E, Whyard S, Baldet T, Bouyer J. Sex Sorting for Pest Control: It's Raining Men! Trends in Parasitology. 2019. pp. 649-662. doi:10.1016/j.pt.2019.06.001
44. Theilmann D A, Stewart S. Molecular analysis of the trans-activating IE-2 gene of Orgyia pseudotsugata multicapsid nuclear polyhedrosis virus. Virology. 1992; 187: 84-96.
45. Carballar-Lejarazu R, Ogaugwu C, Tushar T, Kelsey A, Pham T B, Murphy J, et al. Next-generation gene drive for population modification of the malaria vector mosquito, Anopheles gambiae. Proc Natl Acad Sci USA. 2020; 117: 22805-22814.
46. Gratz S J, Rubinstein C D, Harrison M M, Wildonger J, O'Connor-Giles K M. CRISPR-Cas9 Genome Editing in Drosophila. Curr Protoc Mol Biol. 2015; 111: A.4.1.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method for gene drive propagation, comprising utilizing a modified nickase to introduce paired-nicks to produce single-strand DNA breaks to allow the propagation of an engineered genetic element.

2. The method of claim 1, wherein the nickase is nickase Cas9.

3. The method of claim 2, wherein said nickage Cas9 introduces paired-nicks and promote homology-directed repair (HDR) in a germline of a living organism.

4. A nickase-based gene-drive system comprising a modified nickase to introduce paired-nicks to produce single-strand DNA breaks to allow the propagation of an engineered genetic element in a living organism.

5. The nickase-based gene-drive system of claim 4, wherein said nickase is nickase Cas9.

6. The nickase-based gene-drive system of claim 5, wherein said nickage Cas9 introduces paired-nicks and promote homology-directed repair (HDR) in a germline of the living organism.

7. The nickase-based gene-drive system of claim 4, wherein said living organism is Culex quinquefasciatus, Anopheles mosquitoes, Aedes mosquitoes, Drosophila suzukii fruit fly, pest or insect.

8. The nickase-based gene-drive system of claim 7, wherein doublesex genes in females are disrupted to suppress mosquito species.

9. A method for developing a suppression gene drive system in Culex quinquefasciatus (Cx.quin.) comprising the method of claim 1.

10. The method of claim 9, wherein said method comprising:
a) building and validating constructs with promotor sequences suitable to drive an expression of CRISPR components of Cas9 protein and gRNA, respectively;
b) developing targeted transgenesis of a ubiquitous Cas9 line; and
c) engineering the species and a gene drive construct for population suppression.

11. The method of claim 10, wherein the promoters for expression Cas9 are ubiquitous promotor/E1 and Actin5C and/or germline-specific promoter vas (vasa) and zpg (zero-population-growth).

12. The method of claim 10, wherein the promoters for expression gRNAs are Pol-II promoters driving U6-small-nuclear-RNAs.

13. The method of claim 10, wherein a gRNA sequence targeting a white gene of Cx. quin. is cloned.

14. The method of claim 10, wherein step b) comprises a step of developing targeted HDR-based delivery of transgenic elements in Cx. quin.

15. The method of claim 14, wherein a Cx. quin. Cas9 line comprising a ubiquitous-Cas9-expressing is generated.

16. The method of claim 14 wherein a Cx. quin. gene-drive line comprising a construct comprising Cas9, a gRNA, and a marker gene for population suppression is generated.

17. The method of claim 16, wherein the construct is inserted on one of doublesex (dsx) exons common to both sexes' isoforms, providing a re-coded version of dsxM transcript.

18. The method of claim 16, wherein two closely located gRNAs in a Protospacer Adjacent Motif (PAM) is used to lead to efficient HDR-conversion when using multiplexing.

19. The method of claim 10, further comprising a step of validating a functionality of Cas9 line by injecting the Cas9 line with a plasmid expressing a gRNA targeting a pigmentation marker, yellow, and evaluating the Cas9 activity of the Cas9 line by analyzing yellow phenotype in G0 injected mosquitos and their G1 offspring.

20. The method of claim 16, further comprising a step of testing an ability of the Cx. quin. gene-drive line to copy on companion chromosome, resulting in gene-drive offspring to be >50% males.
